(19) United States
(12) Patent Application Publication
(10) Pub. No.: US 2023/0264163 A1
(43) Pub. Date: Aug. 24, 2023

(54) SOFTSENSOR FOR MORPHOLOGY OF POLYMERS

(71) Applicants: BASF SE, Ludwigshafen (DE); The University of the Basque Country, Donostia San Sebastian (ES); Polymat Fundazioa, Donostia San Sebastian (ES)

(72) Inventors: Wolfgang Gerlinger, Ludwigshafen am Rhein (DE); Hugo Vale, Ludwigshafen am Rhein (DE); Jose M. Asua, Donostia San Sebastian (ES)

(21) Appl. No.: 18/018,114

(22) PCT Filed: Jul. 26, 2021
(86) PCT No.: PCT/EP2021/070859

§ 371 Date: Jan. 26, 2023

(30) Foreign Application Priority Data
Jul. 27, 2020 (EP) 20187939.2

(51) Int. Cl. B01J 19/00

(52) U.S. Cl. CPC B01J 19/0033; B01J 2219/002; B01J 2219/00202

(57) ABSTRACT

A method of producing a soft sensor for a reference morphology of multiphase latex polymer particles synthesized in a production process is provided. The method is used in monitoring and/or controlling the production process. The method may also be used for optimizing production capacities of the production process.

### FIELD

The invention relates to a method of providing a soft sensor for a reference morphology, a system for providing a soft sensor for a reference morphology, a system for providing a soft sensor for a reference morphology and a computer program product for providing a soft sensor. The invention also relates to a soft sensor for a reference morphology. The invention further relates to a method monitoring and/or controlling morphology of multiphase latex polymer particles synthesized in a production process.

### BACKGROUND

Polymerization is an important process in chemical industries for production of various materials. Emulsion polymerization is an important subclass of polymer chemistry, because of the wide application range of the dispersions of polymer particles formed in the emulsion polymerization process. Polymer particles containing more than one phase are extensively used to improve performance and to meet requirements that cannot be achieved by single-phase particles. The physical and chemical properties of polymer particles formed in emulsion polymerization are highly dependent on the morphology of the polymer particles. It is known that the morphology of polymer particles depends on the reaction conditions.

There is a need for an improved method of controlling the polymer particle morphology in emulsion polymerization.

### DESCRIPTION

In order to address the above-mentioned problems, the following method is proposed:

A method of producing a soft sensor for a reference morphology of multiphase latex polymer particles synthesized in a production process, for use in monitoring and/or controlling the production process and/or optimizing production capacities of the production process, comprising the steps of:
providing via an interface to a processing device;
   time series data from a reference polymerization process,
   a morphology functional, wherein the morphology functional describes the movement of polymer clusters in multiphase latex polymer particles since the instance of the polymer cluster-formation along a reaction progression, in a reaction process, by relating a movement of polymer clusters in multiphase latex polymer particles since the instance of polymer cluster formation to time series data of a reaction process
determining at the processing device
   a reference trajectory of the morphology functional up to a reference observation point along the reference reaction progression based on
      the time series data from the reference polymerization process, and
      the morphology functional,
providing the soft sensor, the soft sensor comprising
   a reference trajectory of the morphology functional determined from the reference polymerization process,
   the morphology functional and
   a sensor input for receiving time series data from a production process of the multiphase latex polymer particles
   an output for
      a) the reference trajectory of the morphology functional and the production trajectory of the morphology functional for the production process or
      b) a deviation between the reference trajectory and the production trajectory.

The time series data from the reference polymerization process may comprise a temperature of a reference reactor, flowrates of each ingredient fed into the reference reactor, and the time series data for the production process may comprise a temperature of a production reactor flowrates of each ingredient fed into the production reactor.

The respective time series data may further comprise the respective initial amount of each ingredient fed into the respective reactor.

The respective time series data may further comprise data suitable for determining the reaction progression variable of the production process, and the quantity indicative of a polymer and/or monomer content inside the polymer matrix in the production process. This is in particular useful, when the morphology functional depends on a quantity indicative of a polymer and/or monomer content inside the polymer matrix in the reaction process and a reaction progression variable.

In a perspective aspect a system for producing a soft sensor for a reference morphology of multiphase latex polymer particles synthesized in a production process is provided. The system comprising an input and an output interface, the system further comprising a processor configured for performing the method steps of the method of producing the soft sensor disclosed herein is proposed.

In a perspective a computer program product for producing a soft sensor for a reference morphology of multiphase latex polymer particles synthesized in a production process, for use in monitoring, controlling the production process or optimizing production capacities of the production process that when run on a processor performs the method steps of the method of providing the soft sensor disclosed herein is proposed.

In a perspective a non-transitory computer-readable storage medium is proposed, the computer-readable storage medium including instructions that when executed by a computer, cause the computer perform the steps of the method of providing the soft sensor as disclosed herein.

In a perspective a soft sensor for monitoring and or controlling a polymerization process is provided the soft sensor comprising
a reference trajectory of the morphology functional derived from a reference polymerization process,
a morphology functional describing the movement of polymer clusters in multiphase latex polymer particles since the instance of the polymer cluster formation along a reaction progression, in a reaction process, by relating a movement of polymer clusters in multiphase latex polymer particles since the instance of polymer cluster formation to time series data of a reaction process and
an input for receiving time series data from a production process of the multiphase latex polymer particles, an output for the reference trajectory of the morphology functional and a production trajectory of the morphology functional for the production process, wherein the production trajectory of the morphology functional may be determined based on the time series data of the reaction process and the morphology functional.

In an aspect the time series data from a production process of the multiphase latex polymer particles may comprise
temperature of the production reaction reactor and
flowrates of each ingredient fed into the production reactor and
an initial amount of the monomers of the production process.
a quantity indicative of a polymer and/or monomer content inside the polymer matrix
an output for the reference trajectory of the morphology functional and a production trajectory of the morphology functional for the production process, wherein the production trajectory of the morphology functional may be determined based on the time series data of the reaction process and the morphology functional.

In a perspective a method for monitoring and/or controlling the morphology of multiphase latex polymer particles synthesized in a production process is provided. The method comprising the steps of
providing the soft sensor
   providing time series data from a production process to an input of the soft sensor, to the input interface of the soft sensor
      determining via the soft sensor the production trajectory of the morphology functional
         providing the reference trajectory of the morphology functional and the production trajectory of the morphology functional to a controller unit.

In an aspect the time series data from the production process may comprise
a temperature of a production reactor,
flowrates of each ingredient fed into the production reactor and an initial amount of monomers of the production process, to the input interface of the soft sensor
a quantity indicative of a polymer and/or monomer content inside the polymer matrix

In an aspect a computer program product for monitoring and/or controlling the morphology of multiphase latex polymer particles synthesized in a production process, that when run on a processing device performs any of the method steps according to the method of monitoring and/or controlling as disclosed herein is proposed.

In an aspect system for monitoring and/or controlling the morphology of multiphase latex polymer particles synthesized in a production process comprising a processor an input interface and an output interface, wherein the processor is configured for performing the method steps according to the method of monitoring and/or controlling as disclosed herein is proposed.

In a perspective a system comprising
a monomer suitable for emulsion polymerization
and a soft sensor comprising
   a reference trajectory of the morphology functional derived from a reference polymerization process,
   a morphology functional describing the movement of polymer clusters in multiphase latex polymer particles since the instance of the polymer cluster formation along a reaction progression, in a reaction process, by relating a movement of polymer clusters in multiphase latex polymer particles since the instance of polymer cluster formation to the time series data of the reaction process and
   an input for receiving time series data from a production process of the multiphase latex polymer particles,
   an output for the reference trajectory of the morphology functional and a production trajectory of the morphology functional for the production process is proposed.

In a perspective method of providing a soft sensor for a reference morphology of multiphase latex polymer particles synthesized in a production process, for use in monitoring, controlling the production process or optimizing production capacities of the production process, is proposed, the method comprising the steps of:
providing via an interface to a processing device;
   a morphology functional, wherein the morphology functional describes the movement of polymer clusters in multiphase latex polymer particles since the instance of the polymer cluster-formation along a reaction progression, in a reaction process,
   time series data from a reference polymerization process,
determining at the processing device
   a reference trajectory of the morphology functional up to a reference observation point along the reference reaction progression based on the time series data from the reference polymerization process,
providing the reference trajectory of the morphology functional via an output interface.

In a perspective aspect a system for providing a soft sensor for a reference morphology of multiphase latex polymer particles synthesized in a production process is provided. The system comprising an input and an output interface, the system further comprising a processor configured for performing the method steps of the method of producing the soft sensor disclosed herein is proposed.

In a perspective a computer program product for providing a soft sensor for a reference morphology of multiphase latex polymer particles synthesized in a production process, for use in monitoring, controlling the production process or optimizing production capacities of the production process that when run on a processor performs the method steps of the method of providing the soft sensor disclosed herein is proposed.

In a perspective a soft sensor for a reference morphology of multiphase latex polymer particles synthesized in a production process, comprising a reference trajectory of the morphology functional of a morphology functional up to a reference observation point along the reference reaction progression, wherein the reference trajectory of the morphology functional is determined based on
a morphology functional, wherein the morphology functional describes the movement of polymer clusters in multiphase latex polymer particles since the instance of the polymer cluster formation along a reaction progression, in a reaction process, and
time series data from a reference polymerization process is disclosed.

In a perspective a method for monitoring and/or controlling the morphology of multiphase latex polymer particles synthesized in a production process is provided. The method comprising the steps of
using a reference trajectory of the morphology functional, wherein the reference trajectory of the morphology functional is determined based on
   a morphology functional, wherein the morphology functional describes the movement of polymer clusters in multiphase latex polymer particles since the instance of the polymer cluster formation along a reaction progression, in a reaction process,
   time series data from a reference polymerization process
providing a monitoring and/or control signal via an output interface.

In an aspect a computer program product for monitoring and/or controlling the morphology of polymer particles of a production process in emulsion polymerization in particular in a second or later stage of the emulsion polymerization in a reactor, that when run on a processing device performs any of the method steps according to the method of monitoring and/or controlling as disclosed herein is proposed.

In a perspective a non-transitory computer-readable storage medium is proposed, the computer-readable storage medium including instructions that when executed by a computer, cause the computer to perform the steps of the method of controlling/monitoring the morphology of polymer particles of a production process as disclosed herein.

In an aspect system for monitoring and/or controlling the morphology of multiphase latex polymer particles synthesized in a production process comprising a processor an input interface and an output interface, wherein the processor is configured for performing the method steps according to the method of monitoring and/or controlling as disclosed herein is proposed.

In a perspective a method of optimizing the capacity of a production process while maintaining a reference morphology, comprising the steps of providing constraints to a processing device, determining with the processing device an optimal production capacity based on a reference trajectory of the morphology functional, wherein the reference trajectory of the morphology functional is based on
a morphology functional, wherein the morphology functional describes the movement of polymer clusters in multiphase latex polymer particles since the instance of the polymer cluster formation along a reaction progression, in a reaction process,
time series data from a reference polymerization process is provided.

In a perspective a system for optimizing capacity of a production process while maintaining a reference morphology comprising a processor an input interface and an output interface, wherein the processor is configured for performing the method steps of optimizing capacity of a production process while maintaining a reference morphology as disclosed herein is provided.

In a perspective a computer program product for optimizing capacity of a production process while maintaining a reference morphology, that when run on a processing device performs method steps according the method steps of optimizing capacity of a production process while maintaining a reference morphology as disclosed herein is provided.

In a perspective a system comprising
a monomer suitable for emulsion polymerization
and a soft sensor, comprising a reference trajectory of the morphology functional of a morphology functional up to a reference observation point along the reference reaction progression, wherein the reference trajectory of the morphology functional is determined based on
   a morphology functional, wherein the morphology functional describes the movement of polymer clusters in multiphase latex polymer particles since the instance of the polymer cluster formation along a reaction progression, in a reaction process,
   time series data from a reference polymerization process is proposed.

A production process is understood as the polymer reaction process to be controlled in accordance with the present disclosure.

In an aspect the reference morphology relates to a reference morphology of latex polymer particles, wherein the latex polymer particles are multiphase latex polymer particles. In an aspect, the latex polymer particles are synthesized in a reference polymerization process. The morphology functional describes the movement of polymer clusters in multiphase latex polymer particles since the instance of the polymer cluster formation along a reaction progression This may be realized, by relating a movement of polymer clusters in multiphase latex polymer particles since the instance of polymer cluster formation to temperature of a reaction reactor, flowrates of each ingredient fed into the reactor, a quantity indicative of a polymer and/or monomer content inside the polymer matrix and an initial amount of the monomers. The movement may be understood as a travelled distance.

This has the advantage that a comprehensible representation of the evolution of morphology is provided as a function of reaction progression. This has the advantage of relating measurable and controllable reaction process variables parameters of the production polymerization process to a morphology of a reference polymerization process, which enables reproducible production of polymers. This further allows monitoring and/or controlling of the production process. This allows a convenient method of describing reaction conditions leading to the same morphology of the multiphase latex polymer particles based on measurable reaction process variables that can be controlled during a reaction process, this enables controlling.

Furthermore, using the morphology functional no longer requires complex real time simulations of the dynamics of polymer formation, which allows a faster control of the process due to reduced lag times in a control loop.

Achievement of a desired morphology, e.g. the morphology from a reference polymerization process is key in providing polymers with consistent quality, currently this is mostly performed by maintaining identical operating variables of the reaction process. Making it difficult to reduce production times. By associating the morphology of the particle to the reaction progression via the morphology functional, the production time can be reduced without changing the morphology. Leading to optimized production processes with constant quality. Due to the complex nature of emulsion polymerization, and the interdependence between reaction speed and morphology it is currently not possible.

Furthermore, the soft sensor enables maintaining a desired morphology independent of the plant design in which the reaction is performed. In chemical industries experiments are often run in small laboratory environment and will then need to be transferred to a larger scale production once a desired morphology can be achieved in experiments. Due to the complex nature of emulsion polymerization, and the influence of the experimental setup on morphology it is currently difficult to maintain a desired morphology when transferring between different setups. Therefore, generally pilot plants are designed and built as an intermediate step. This is very costly as the reaction parameters are generally not scalable from lab to pilot to production.

The next paragraphs provide some insight on the understanding of terms used throughout the description. While generally disclosed in relation to a general reaction process, it is understood that the terms explained in relation to a general reaction process may be used for the reference reaction process and the product process alike.

The reference reaction process may in other words be a reference polymerization process.

Time series data is generally understood as a set of data collected sequentially. In some aspect the data may be collected at fixed intervals of time. In some aspect the data may be collected with a time stamp. In some examples the time series data only comprises one specific datum provided at one or more times.

Polymer clusters in this disclosure are understood as polymerized monomers. More particular clusters may be polymerized monomers which are in contact with the polymer matrix of the multiphase latex polymer particle. Even more particular polymer clusters may further be understood as aggregates of macromolecules in other words polymer chains [L.J González-Ortiz, J.M. Asua, Development of Particle Morphology in Emulsion Polymerization. 1. Cluster Dynamics, Macromolecules 1995, 28, 9, 3135-3145]. Methods for precise characterization of polymer clusters are available [Coupling HAADF-STEM Tomography and Image Reconstruction for the Precise Characterization of Particle Morphology of Composite Polymer Latexex Noushin Rajabalinia, Shaghayegh Hamzehlou, Evgeny Modin, Andrey Chuvilin, Jose R. Leiza, José M. Asua Macromolecules 2019, 52, 5298-5306]. More particular, polymer clusters are understood as second stage monomers polymerized in the multiphase latex polymer particle and the resulting polymer phase separated from the polymer matrix.

The term “in contact” may be understood as being influenced by the polymer particle matrix, for instance by being attached to the surface of the polymer matrix or by being partly or fully embedded in the polymer matrix. Polymer clusters in contact with the polymer matrix are understood as part of the multiphase latex polymer particle in other words “in” the polymer particle. The terms “in” or “inside” when referred to the polymer clusters relative to the multiphase latex polymer particle is understood as clusters being influenced by the polymer matrix of the multiphase latex polymer particle.

Multiphase latex polymer particles are understood as particles comprising more than one phase. One phase in the multiphase latex polymer may be the polymer particle matrix. Morphology of multiphase latex polymer particles is understood as mean three-dimensional shape of polymer of the multiphase latex polymer particles. This accounts for the fact that each of the multiphase latex polymer particles may have a slightly different shape, but a characteristic mean overall shape may be identified.

The reference polymerization process is understood as a reaction process in emulsion polymerization in particular in a second or later stage of the emulsion polymerization.

The term “reaction temperature” may be understood as the temperature in the reactor.

The term “soft sensor” may relate to a sensor for monitoring or controlling a polymerization process. Soft sensors generally provide inputs for receiving directly measured physical quantities and provide these to an internal model, that relates the physical quantities to a measure that is not directly accessible. The soft sensor may comprise the reference trajectory of the morphology functional, the morphology functional and an input for receiving time series data from a production process of the multiphase latex polymer particles, comprising temperature of a reaction reactor and flowrates of each ingredient fed into the production reactor and an initial amount of the monomers of the production process.

In an aspect the time series data may further comprise data suitable for determining the reaction progression variable of the reaction process, and the quantity indicative of a polymer and/or monomer content inside the polymer matrix in the reaction process.

In an aspect the reference polymerization process may be a real reaction process or an in-silico reaction process. In this description real reaction means reaction in a physical experiment.

In an aspect the production process may be a real reaction process or an in-silico reaction process. A real reaction process may be advantageous, for controlling. An in-silico reaction process may be advantageous for process optimization.

Applying the proposed methods to in silico reaction processes allows to speed up developments as no experiments are required. This saves costs and time in finding operating conditions.

The trajectory of the morphology functional up to an observation point along the reaction progression describes the movement of polymer clusters in multiphase latex polymer particles since the instance of the polymer cluster-formation up to the observation point for all polymer clusters created along the reaction progression of the reaction until the observation point. This is a measure for the travelled distance of polymer clusters in multiphase latex polymer particles since the instance of polymer cluster formation up to the observation point for all polymer clusters created along the reaction progression of the reaction until the observation point.

For determining at the processing device, the trajectory of the morphology functional up to a reference observation point along the reference reaction progression may be based on the time series data from the reference polymerization process. For determining the reference trajectory of the morphology functional, the times series data of the reference reaction are provided.

In an aspect, the morphology functional may depend on
a quantity indicative of a polymer and/or monomer content inside the polymer matrix in the reaction process and
a reaction progression variable.

This means that the morphology functional may depend on the temperature of a reactor, flowrates of each ingredient fed into the reactor and an initial amount of the monomers

The reaction progression variable describes how far the reaction has progressed.

In an aspect, the reaction progression variable may be a quantity indicative of the amount of polymerized monomers in the reaction process.

The quantity indicative of the amount of polymerized monomers in the reaction process provides information on the reaction progression. Furthermore, the quantity indicative of the amount of polymerized monomers in the reference polymerization process and the amount of monomer added to the reactor provide information on the amount of unreacted monomers.

In an aspect the quantity indicative of the amount of polymerized monomers in the reaction process may be directly measured, by measuring the amount of polymerized monomers in the reactor or determined from the amount of unreacted monomers in the reactor.

The quantity indicative of a polymer and/or monomer content inside the polymer matrix has the advantage that it is a well-known concept in polymer chemistry and can easily be determined, so providing the morphology functional dependent on the quantity indicative of a polymer and/or monomer content inside the polymer matrix in the reaction process allows easy further processing of the morphology functional.

In an aspect the reaction progression variable is time, in particular the time since the reaction started. In some instances, it may be preferable to describe the reaction progression in terms of a normalized time, e.g. the ratio between the time elapsed since the start of the polymerization and the total polymerization time.

This has the advantage that time can be easily measured and therefore, the morphology functional be easily determined from the time series data

In an aspect the reaction progression variable may reflect conversion of monomers into polymers.

In an aspect the reaction progression variable may be an overall monomer conversion.

This has an advantage that the reaction progression variable is independent of reaction time.

In an aspect the morphology functional may be a function of the overall monomer conversion. This has the advantage that the morphology functional is independent of reaction time. In other words, a fast reaction may lead to a similar morphology as a slow reaction, if the morphology functional provides similar values for the same overall monomer.

This means that the morphology functional may correctly describe the evolution of particle morphology in different polymerization reactors and different operating conditions. Therefore, the same morphology functional may be used to compare and control the evolution of particle morphology in different production setups, differing for instance in geometry (e.g. reactor size) or reaction conditions (e.g. ingredient flowrates, temperature, etc.). This is in particular useful when a desired polymer product with a certain particle morphology is to be produced in different polymerization reactors and/or different operating conditions.

In an aspect, the morphology functional may comprise a cluster mobility function, describing a mobility of the polymer clusters in the latex polymer particles during progression of the reaction process by relating a movement of polymer clusters in multiphase latex polymer particles since the instance of polymer cluster formation to time series data of a reaction process.

The evolution of polymer particle morphology in a reaction process is based on the movement of polymer clusters formed during progression of the reaction. The movement may depend on driving and drag forces on the polymer clusters. The driving forces for this movement may be interfacial tensions and van der Waals forces in the interior of the polymer particle, both of them are expected not to vary significantly from run to run if either the same polymers or polymers of similar hydrophobicity, and similar surfactants are used. The interior of the polymer particles may be the monomer swollen seed polymer often called also called polymer matrix.

In an aspect, the cluster mobility function may depend on the quantity indicative of a polymer and/or monomer content inside the polymer matrix in the reaction. The advantages of the dependent variables were already described above with respect to the morphology functional.

In an aspect the time series data may comprise data suitable for determining
the reaction progression variable,
the quantity indicative of a polymer and/or monomer content inside the polymer matrix in the reference polymerization process.

This means that the morphology functional may depend on the temperature of a reactor, flowrates of each ingredient fed into the reactor and an initial amount of the monomers

When the functional relationships depend on these inputs it is favorable to provide these in the time series data such that the step of determining may be performed.

In an aspect the morphology functional may depend on a temperature in the reactor and the time series data may comprise a temperature in the reference reactor.

This allows evaluation of non-isothermal processes. The temperature in the reactor may have an influence on the reaction, therefore it is beneficial when the time series data comprises the temperature in the reference reactor.

In an aspect the morphology functional may depend on a glass transition temperature of the seed and the time series data may comprise data on the glass transition temperature of the seed.

The dependency on the glass transition temperature of the seed allows transferring of the cluster mobility function from one polymer system to another polymer system with similar glass transition temperatures.

In an aspect, the morphology functional may further depend on molecular weight of the seed. The dependency on the molecular weight of the seed allows transferring of the cluster mobility function from one polymer system to another polymer system.

The glass transition temperature of a seed can be seen as a description how “hard” the polymer seed is at a given temperature. Temperatures are easily measurable and glass transition temperatures of the seed can be determined.

In an aspect the cluster mobility function may comprise a function of a particle matrix viscosity.

Using a function of a particle matrix viscosity in describing the mobility function has the advantage, that particle matrix viscosities are often known or can be determined by experiments.

In an aspect the particle matrix viscosity may be a relative matrix viscosity.

Absolute values for the matrix viscosity are sometimes difficult to determine. Introducing the relative matrix viscosity, eliminates the need of knowledge of absolute values of the particle matrix viscosity.

In an aspect, the relative particle viscosity may depend on the temperature in the reactor and a quantity indicative of the polymer and/or monomer content inside the polymer matrix in the reaction process. In that case, the time series data may comprise the temperature of the reactor.

In an aspect the reference trajectory of the morphology functional is determined by determining the movement of polymer clusters in multiphase latex polymer particles since the instance of polymer cluster formation along a reaction progression, in a reaction process until an observation point and arranging along the progression variable in the order of instance of formation of the polymer clusters for all instances of formation.

The method of monitoring and or controlling provides a simple way of monitoring and/or controlling the morphology.

The morphology of multiphase latex polymer particles is important for the properties of the polymer. The difficulties of monitoring and/or controlling the morphology during the reaction is overcome by using the soft sensor for control. Reactions following the reference trajectory of the morphology functional will lead to a similar morphology as in the reference polymerization process. Therefore, monitoring and/or controlling may enabled by following the reference trajectory of the morphology functional. Following the reference trajectory of the morphology functional may be realized by various control algorithms. The reference trajectory of the morphology functional may be considered a setpoint for control.

Further advantages of the controlling are disclosed below with reference to another method of controlling.

In an aspect the method may further comprise the steps of providing via the interface time series data from a production process and determining a production trajectory of the morphology functional based on the morphology functional, wherein the morphology functional describes the movement of polymer clusters in multiphase latex polymer particles since the instance of the polymer cluster formation along a reaction progression, in the reaction process, and the time series data from the production process.

Providing time series data of the production process may provide information of the reaction evolution. The production trajectory of the morphology functional may then be used as actual value input for monitoring and/or controlling.

In an aspect the time series data may comprise data suitable for determining
the reaction progression variable, and
the quantity indicative of a polymer and/or monomer content inside the polymer matrix in the reference polymerization process.

This means that the time series data may comprise the temperature of a reactor, flowrates of each ingredient fed into the reactor and an initial amount of the monomers

In an aspect the morphology functional may further depend on the reaction temperature.

In an aspect the time series data may further comprise the temperature in the production reactor. The temperature in the reactor is easy to measure.

In an aspect the morphology functional may further depend on, flowrates of each ingredient fed into the reactor

In an aspect the morphology functional may further depend on an initial amount of the monomers.

In an aspect the morphology functional may further depend on a glass transition temperature of the seed.

In an aspect the time series data may further comprise the glass transition temperature of the seed in the production reactor.

In an aspect the morphology functional may further depend on a molar mass of a seed.

In an aspect the time series data may further comprise the molar mass of the seed in the production reactor.

In an aspect the cluster mobility function may further comprise a function of a particle matrix viscosity.

In an aspect the reference trajectory of the morphology functional is determined by determining
the movement of each polymer cluster in multiphase latex polymer particles since the instance of the polymer cluster formation along a reaction progression, in a reaction process until an observation point and
arranging along the progression variable in the order of instance of formation of the polymer clusters for all instances of formation.

In an aspect the cluster mobility function is the same for the reference polymerization process and the production process.

The time series data for optimizing may comprise time series data from a real reaction process or an in-silico reaction process.

In an aspect the time series data may comprise data suitable for determining
the reaction progression variable, and
the quantity indicative of a polymer and/or monomer content inside the polymer matrix in the reference polymerization process.

In particular, these time series data may comprise
a temperature of a reference reactor,
flowrates of each ingredient fed into the reference reactor; and
an initial amount of monomers of the reference polymerization process.

From these time series data, the reaction progression variable and the quantity indicative of a polymer and/or monomer content inside the polymer matrix in the reference polymerization process can be determined.

In an aspect the morphology functional may further depend on the reaction temperature. The reaction temperature may be understood as the temperature in the reactor.

In an aspect the time series data may further comprise the temperature in the production reactor. The temperature in the reactor is easy to measure.

In an aspect the morphology functional may further depend on a glass transition temperature of the seed.

In an aspect the time series data may further comprise the glass transition temperature of the seed in the production reactor.

In an aspect the morphology functional may further depend on a molar mass of a seed.

In an aspect the time series data may further comprise the molar mass of the seed in the production reactor.

In an aspect the cluster mobility function may further comprise a function of a particle matrix viscosity.

Providing the soft sensor has the advantage, that the step of determining the reference trajectory of the morphology functional may be provided in another system either ahead of the production run or as a service. In that scenario the method of providing the reference time series data may comprise providing the reference time series data via a web interface or an API.

Providing the monomer together with the soft sensor provides the customer with information on achieving a desired morphology and allows easy customization on a specific reaction design.

In an emulsion polymerization, ingredients are provided to the reactor. The ingredients provided to the reactor generally comprise an amount of at least one type of ingredients, which may be monomers provided to the reactor. The monomers will then polymerize.

In an aspect, the at least one monomer comprises a monomer that can be polymerized by radical polymerization. Examples of monomers include but are not limited to esters of acrylic acid and methacrylic acid of formula CH₂═CR²—COOR³ wherein R² is H or methyl and R³ is optionally substituted alkyl or cycloalkyl of 1 to 20 carbon atoms (more preferably 1 to 8 carbon atoms) examples of which are methyl acrylate, methyl methacrylate, ethyl acrylate, ethyl methacrylate, trifluorethyl(meth)acrylate, n-butyl acrylate, n-butyl methacrylate, 2-ethylhexyl acrylate, 2-ethylhexyl methacrylate, isopropyl acrylate, isopropyl methacrylate, and hydroxyalkyl (meth)acrylates such as 2-hydroxyethyl acrylate, 2-hydroxyethyl methacrylate, and their modified analogues. Other esters of acrylic acid and methacrylic acid are those where the alkyl chain is obtained from biosourced feedstocks such as terpenoids or alkyds. Suitable monomers also include 1,3-butadiene, isoprene, styrene, α-methyl styrene, divinyl benzene, acrylamide, acrylonitrile, methacrylonitrile, vinyl halides such as vinyl chloride, vinyl esters such as vinyl acetate, vinyl propionate, vinyl laurate, and vinyl esters of versatic acid such as VeoVa 9 and VeoVa 10 (VeoVa is a trademark of Hexion), heterocyclic vinyl compounds. Unsaturated monocarboxylic and/or dicarboxylic acids, such as acrylic acid, methacrylic acid, fumaric acid, and itaconic acid are other examples that can be used. Other olefinically unsaturated monomers that can be used are those monomers that contain a fatty acid derived ester group such as oleyl (meth)acrylate, linoleyl (meth)acrylate, and linolenyl (meth)acrylate.

In an aspect the ingredients provided to the reactor may comprise more than one type of monomer. Some polymer reactions rely on more than one type of monomers. The use of more than one type of monomers has the advantage that a larger variety of polymers also known as copolymers may be formed. This provides access to a larger variety of properties of the polymer.

In an aspect the ingredients provided to the reactor may further comprise at least one initiator. Initiators are increasing the rate of polymerization. Initiators may increase the rate of polymerization by generating free radicals by thermal decomposition, or redox reactions. Suitable free-radical polymerization initiators fundamentally include both inorganic peroxides and hydroperoxides, e.g., hydrogen peroxide, peroxodisulfates such as sodium peroxodisulfate, ammonium peroxodisulfate, and organic peroxides or hydroperoxides, e.g., tert-butyl hydroperoxide, and azo compounds. Examples of suitable initiators are sodium peroxodisulfate or ammonium peroxodisulfate. Preference is also given to redox initiator systems which are composed of at least one organic reducing agent and at least one peroxide and/or hydroperoxide. Examples thereof are combinations of tert-butyl hydroperoxide with ascorbic acid or with at least one sulfur compound, e.g., the sodium salt of hydroxymethanesulfinic acid, sodium sulfite, sodium disulfite, sodium thiosulfate or acetone bisulfite adduct; or combinations of hydrogen peroxide with ascorbic acid or with at least one of the abovementioned sulfur compounds. The redox initiator systems may further include a small amount of a metal compound which is soluble in the polymerization medium and whose metallic component is able to exist in a plurality of valence states; iron(II) sulfate together if desired with EDTA, vanadium salts, etc. The amount of the free-radical initiator systems used, based on the total amount of the monomers for polymerization, is preferably from 0.1 to 5% by weight.

The ingredients provided to the reactor may further comprise a surfactant. Examples of ionic surfactants include but are not limited to alkyl sulfates such as sodium lauryl sulfate, alkyl-aryl sulfonates such as sodium dodecylbenzene sulfonate, polyoxyethylene alkyl ether sulfates, polyoxyethylene alkylphenyl ether sulfates, and diphenyl sulfonate derivatives. Examples of non-ionic surfactants include but are not limited to polyoxyethylene alkyl ethers or polyoxyethylene alkylphenyl ethers.

Electrosteric surfactant have both ionic and non-ionic stabilizing moieties. Examples of electrosteric surfactants include but are not limited to sodium alkyl polyether sulfonates and alkali soluble resins.

Examples of polymeric surfactants include but are not limited to poly(vinyl alcohol) and diblock and tri-block copolymers of poly(ethylene glycol) and poly(propylene glycol) such as those of the Pluronics™ series (BASF).Examples of reactive surfactants include, but are not limited to E-Sperse™ series (Ethox Chemicals), Visiomer™ series (Evonik) and Maxemul™ series (Croda),

In an aspect, the ingredients provided to the reactor may comprise a continuous medium or a seed.

Examples of continuous medium include but are not limited to water, organic solvents, ionic liquids and supercritical liquids.

A seed is a dispersion of polymer particles in a continuous medium. In the seed the polymer can be produced by chain growth polymerization. In the seed the polymer can also be produced by step growth polymerization. Furthermore, the seed can be synthesized by combining step growth and chain growth polymerizations.

In an aspect the production of the seed may be in the first stage of the polymerization.

In an aspect the ingredients provided to the reactor may comprise at least one inhibitor. Inhibitors are mainly used to stabilize monomers for increasing shelf life.

In an aspect the ingredients provided to the reactor may comprise an initial amount of ingredients provided at a start of the reaction process Mᵢ₀. Wherein Mᵢ₀ is the initial amount for each ingredient and i is an index for each type of ingredient.

The initial amount of ingredients provides relevant data regarding the start of the reaction.

In an aspect the ingredients provided to the reactor may comprise an amount of ingredients provided during the reaction.

In an aspect the amount of ingredients provided during the reaction may be the integral of the ingredients flow rate for each ingredient.

In an aspect the amount of ingredients provided during the reaction may be measured by flow sensors for each of the ingredients. This may be described as measured flowrates of all ingredients added to the reactor throughout the entire course of the polymerization: ∀ᵢ, Fᵢ(t), 0 ≤ t ≤ t_{end}. Wherein Fᵢ is the flow rate for each ingredient and i is an index for each type of ingredient.

This is in particular useful for semi-batch production, where the flow of ingredients into the reactor may vary over time.

In an aspect the amount of ingredients provided to the reactor may comprise an initial amount of ingredients provided at a start of the reaction process and the amount of ingredients provided during the reaction. In that case the full amount of ingredients for each ingredient the reaction is known at all points in the reaction.

The amount of ingredients provided to the reactor, the ingredients comprising at least one type of monomer, may relate to any suitable unit of measurement for each of the ingredients (e.g. moles, mass, volume).

The time series data, suitable for determining
a quantity indicative of the polymer or monomer content inside the polymer matrix in the reaction process and
a reaction progression variable
may for example comprise at least one of the list of, a temperature of the reactor; a cooling/heating jacket temperature; flowrates of each ingredient fed into the reactor; temperatures of each ingredient fed into the reactor; an initial amount of each ingredient in the reactor; a flowrate of a cooling medium in the cooling/heating jacket; physicochemical analysis spectroscopic data (Raman, IR, NMR, etc.); reactor pressure; surrounding temperature; stirrer torque and stirrer rotation speed.

These examples are non-limiting. The term cooling/heating jacket refers to a jacket surrounding a reactor, that is capable of cooling and/or heating.

The time series data comprising the temperature of the reactor may be sensor data. In particular, the sensor may be a temperature sensor. Various suitable different temperature sensors are known in the art (e.g. thermocouples, thermistors, resistance thermometer, silicon bandgap temperature sensors, pyrometers).

In an aspect the time series data of the reference polymerization process may be determined online (e. g. while the reference reaction takes place) or offline (e.g. from samples taken while the reference reaction takes place and analyzed after the reference reaction took place).

In this aspect, the time series data suitable for determining is understood as time series data directly being indicative of the amount of polymerized monomer.

Suitable methods for determining the amount of unreacted monomers or the amount of polymerized monomers in the reactor may comprise e.g. online reaction calorimetry (either heat flow calorimetry or heat balance calorimetry), online or offline physicochemical analysis methods such as spectroscopy (Raman, IR, NMR, etc.), chromatography (HPLC, GC, etc.). For the reference reaction in a real experiment the above-mentioned methods may be performed offline.

In an aspect the quantity indicative of the amount of polymerized monomers in the reaction process may be determined from the provided suitable time series data. Determining the quantity indicative of the amount of polymerized monomers in the reaction process has the advantage that the quantity indicative of the amount of polymerized monomers in the reaction process may be used in further calculations without the need to perform the determining step again.

In an aspect, the quantity indicative of the amount of polymerized monomers in the reaction process may be determined from reaction calorimetry. Reaction calorimetry may comprise heat flow calorimetry or heat balance calorimetry.

In an aspect, the quantity indicative of the amount of polymerized monomers in the reaction process may be determined from heat flow calorimetry. Heat flow calorimetry measures the heat flowing across the reactor wall (Q̅_{J}(t)) and quantifying this in relation to the other energy flows within the reactor. In an aspect, the data indicating the amount of unreacted monomers for each of the type of monomers or the amount of polymer may be determined from heat balance calorimetry. The cooling/heating jacket influences the temperature of the reaction process. Heat transferred to the cooling/heating jacket (Q̅_{J}(t)) is measured by monitoring the heat gained or lost by the heat transfer fluid.

An example is shown in equation (1)

$${\dot{Q}}_{J} \left(t\right) = \left\{\begin{matrix}U A \left(T - {T}_{j a c k e t}\right) , \text{or} \\ {F}_{H T F} {c}_{p , H T F} \left({T}_{j a c k e t , o u t} - {T}_{j a c k e t , i n}\right)\end{matrix}\right) \qquad \text{(1)}$$

Wherein U refers to the overall heat transfer coefficient, A refers to the heat transfer area, T refers to the temperature in the reactor and T_{jacket} refers to the average temperature in the cooling/heating jacket, and (T-T_{jacket}) is generally expressed as the logarithmic mean temperature difference.

Wherein F_{HTF} refers to the mass flow of the heat transfer fluid, c_{p,HTF} refers to the specific heat capacity of the transfer fluid, T_{jacket,out} refers to the outlet temperature of the heat transfer fluid and T_{jacket,in} refers to the inlet temperature of the heat transfer fluid.

The mass flow may be determined from the flowrate of the heat transfer fluid.

The temperature of the reactor may be directly measured.

Reaction calorimetry is a suitable method as polymerization reactors are cooled and the temperature of a reactor and the temperature of the cooling/heating jacket may easily be monitored.

The amount of heat produced by polymerization (Q̅) can be determined by means of the heat balance in the reactor

$${m}_{R} {c}_{p R} \frac{d T}{d t} = \dot{Q} + {\sum }_{i} {F}_{i} {c}_{p i} \left({T}_{i} - T\right) - {\dot{Q}}_{j} \qquad \text{(2)}$$

Wherein m_{R} and c_{pR} are the mass and the specific heat capacity of the reactor and Fᵢ and cₚᵢ the mass flow and specific heat capacity of ingredient i that is fed to the reactor at a temperature Tᵢ.

In an aspect data indicative of the amount of monomer converted to polymer may then be determined by equation (3),

$$\left\{M o l e s o f m o n o m e r p o l y m e r i z e d\right\} = \frac{1}{\text{Δ} {Η}_{p o l}} {\int }_{0}^{t} \dot{Q} d {t}^{′} \qquad \text{(3)}$$

Wherein ΔHₚₒₗ is the enthalpy of the polymerization reaction. If more than one type of monomers is used, data indicative of the amount of each monomer i polymerized (M_{i,polymerized}) and the amount of polymer can be obtained by using a modification of equation (3) as described in literature [Gugliotta, L.M., Arotçarena M., Leiza, J.R., Asua, J.M. Estimation of conversion and copolymer composition in semicontinuous emulsion polymerization using calorimetric data, Polymer, 1995, 36, 2019-2013].

When the quantity indicative of the amount of polymerized monomers in the reaction process is determined from heat flow calorimetry or from heat balance calorimetry, the time series data suitable for determining the quantity indicative of the amount of polymerized monomers in the reaction process may comprise at least one of the list of, the temperature of the reactor; the cooling/heating jacket temperature; the flowrates of each ingredient fed into the reactor; the temperatures of each ingredient fed into the reactor; the initial amount of each ingredient in the reactor; the flowrate of a cooling medium in the cooling/heating jacket; the inlet and outlet temperatures of the heat transfer fluid; surrounding temperature; stirrer torque; and stirrer rotation speed.

In an aspect the quantity indicative of the amount of polymerized monomers in the reaction process may be an overall monomer conversion. The overall monomer conversion may describe the amount of polymerized monomers in the total amount of monomers to be fed until the end of the reaction:

$$\begin{matrix}{X}_{o v e r a l l} \left(t\right) = \\ \frac{\left\{m o n o m e r s p o l y m e r i z e d\right\}}{\left\{m o n o m e r s p r o v i d e d t o r e a c t o r u n t i l e n d o f r e a c t i o n\right\}}\end{matrix}$$

Various alternative suitable options of determining the overall monomer are shown of below as equation (4).

$$\begin{matrix}{X}_{o v e r a l l , m a s s} \left(t\right) = \\ \frac{\left\{M a s s o f m o n o m e r s p o l y m e r i z e d\right\}}{\left\{M a s s o f m o n o m e r s p r o v i d e d t o r e a c t o r u n t i l e n d o f r e a c t i o n\right\}} \\ {X}_{o v e r a l l , m o l e} \left(t\right) = \\ \frac{\left\{M o l e s o f m o n o m e r s p o l y m e r i z e d\right\}}{\left\{M o l e s o f m o n o m e r s p r o v i d e d t o r e a c t o r u n t i l e n d o f r e a c t i o n\right\}} \\ {X}_{o v e r a l l , m o l e} \left(t\right) = \\ \frac{\left\{M o l e s o f m o n o m e r s p o l y m e r i z e d\right\}}{{\sum }_{i = M o n o m e r s} \left({M}_{i 0} + {\int }_{0}^{{t}_{e n d}} {F}_{i} d {t}^{′}\right)}\end{matrix} \qquad \text{(4)}$$

In an aspect the quantity indicative of the polymer or monomer content inside the polymer matrix in the reaction process may be determined from the provided suitable time series data. Determining the quantity indicative of the polymer or monomer content inside the polymer matrix in the reaction process has the advantage that the quantity indicative of the polymer or monomer content inside the polymer matrix in the reaction process may be used in further calculations without the need to perform the determining step again.

In an aspect the quantity indicative of the polymer or monomer content inside the polymer matrix in the production reaction process may be directly determined from the time series data provided to the processor.

In an aspect the quantity indicative of the amount of polymerized monomers in the reaction process is derived from the timeseries data and the quantity indicative of the polymer or monomer content inside the polymer matrix is derived from that.

In an aspect the quantity indicative of monomer or polymer content inside the polymer matrix may be a fraction of the amount of unreacted monomers in the polymer particle and the amount of polymer in the polymer particle.

In an aspect the quantity indicative of monomer or polymer content inside the polymer matrix may be expressed as mass fraction of monomer (or polymer), volume fraction of monomer (or polymer), mass or molar concentration of monomer (or polymer) per unit volume, etc.

When the ingredients comprise more than one type of monomer, the amount of unreacted monomers may refer to the sum of all monomers independently of their type.

As a non-limiting example, the quantity indicative of monomer or polymer content inside the polymer matrix can be suitably be represented as the quantity indicative of monomer or polymer content inside the polymer particle. For simplicity this approximation is used throughout the disclosure.

For example the volume fraction polymer in the particles is:

$$\begin{matrix}{\text{ϕ}}_{p o l} \left(t\right) \approx \\ \frac{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}}}{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}} + \frac{\left\{M a s s o f u n r e a c t e d m o n o m e r s i n t h e p o l y m e r p a r t i c l e s\right\}}{\left\{D e n s i t y o f m o n o m e r s\right\}}}\end{matrix} \qquad \text{(5)}$$

The densities of the polymer, seed and monomers may be provided to the processor via a database or by manually entering into a keyboard, wherein the mass of seed is given by the formulation and the densities are known or may be easily determined from experiments.

The quantity indicative of monomer or polymer content inside the polymer matrix may be obtained from the amount of unreacted monomers and the amount of polymer in the reactor solving the monomer partitioning as described in literature [Armitage P.D., de la Cal, J.C., Asua, J.M. Improved methods for solving monomer partitioning in emulsion copolymer systems. J. Appl. Polym. Sci. 1994, 51, 1985-1990].

Simplifications known to those skilled in the art may also be applied. For instance, for many emulsion polymerizations, the amount of monomer dissolved in the continuous medium is very small (negligible) and it can be considered that all the monomer is in the polymer particles. In this case, in equation (5) the {Mass of unreacted monomers in the polymer particles} may be approximated by the (Mass of unreacted monomers).

In an aspect, the quantity indicative of monomers or polymer content inside the polymer matrix may be considered from a quantity describing the amount of polymerized monomers in the amount of monomers fed until the current time in the reaction also known as total instantaneous monomer conversion. The total instantaneous monomer conversion (Xᵢₙₛₜ) is an established quantity [Unzue M.J., Asua, J.M., Semicontinuous miniemulsion terpolymerization. Effect of operation conditions. J. Apply. Polym. Sci., 1993, 49, 81-90].

It is clear to a person skilled in the art to use different notations for quantities consistent across any calculations and how to transform between them.

To illustrate the principle for deriving quantities instead of the exact mathematical equations, descriptive equations are used throughout the description. It is obvious to a person skilled in the art to use different notations for quantities consistent across any calculations. For simplicity where applicable, the disclosure refers to illustrative wording. Where deemed to be useful, references providing examples of the equations are provided.

In an aspect a morphology functional (Y) is provided to the processor, wherein the morphology functional describes the movement of polymer clusters in multiphase latex polymer particles since the instance of the polymer cluster-formation along a reaction progression in a reaction process, the morphology functional depending on
the time series data, suitable for determining
   the quantity indicative of the polymer or monomer content inside the polymer matrix in the reaction process and
   ◯ a reaction progression variable
and may also depend on
   temperature in the reactor,
   glass transition temperature of the seed polymer (T_{g}) and
   molar mass of the seed polymer (M_{w}).

The morphology functional may accordingly be written as Y=Y(Xₒᵥₑᵣₐₗₗ, Φₚₒₗ, T, time)

In an aspect the cluster mobility function may be a function of
the temperature in the reactor and
a quantity indicative of the polymer or monomer content inside the polymer matrix in the reaction process.

The mobility function may additionally depend on
the glass transition temperature of the seed polymer (T_{g}), and
the molar mass of the seed polymer (M_{w})

Both may be time series data.

The cluster mobility function may then be written as Ψ(T(t), Φₚₒₗ(t)).

The temperature in the reactor and a quantity indicative of the polymer or monomer content inside the polymer matrix in the reaction process may be easily determined from the reaction process as explained above.

In an aspect the cluster mobility function may be a function of the reaction temperature and the total instantaneous monomer conversion.

In an aspect the cluster mobility function may be a function of the reaction temperature and the volume fraction of polymers in the polymer particle.

The cluster mobility function may comprise a function of the relative polymer matrix viscosity. Polymer matrix viscosities are models that describe the relative viscosity of the polymer matrix.

In principle, any equation that reliably describes the dependence of particle matrix viscosity with temperature and monomer (or polymer) content inside the particles may be used with the present method. For example, such a function may be a linear or non-linear function of the polymer volume fraction inside the particles Φₚₒₗ and temperature, which can be represented as:

$$η = η \left({ϕ}_{p o l} , T\right) \qquad \text{(6)}$$

or

$$η = η \left({X}_{i n s t} , T\right) \qquad \text{(7)}$$

if the particle matrix viscosity is a function of the reaction temperature and the total instantaneous monomer conversion.

In an aspect the cluster mobility function may then be

$$\text{ψ} = \frac{1}{η \left({ϕ}_{p o l} , T\right)} \qquad \text{(8)}$$

In an aspect the cluster mobility function depends on the effective glass transition temperature (T_{g}) of the polymer matrix.

In an aspect, the particle matrix viscosity may be described as:

$$\left(η \left({ϕ}_{p} , T\right) = A {ϕ}_{p o l}^{n} \text{exp} \left(B \frac{{T}_{g e f f e c t}}{T} - C\right)\right) \qquad \text{(9)}$$

Wherein T_{geffect} is the effective glass transition temperatures of the polymer matrix, and A, n, B and C are parameters that can be determined. The effect of the molar mass of the seed is included in the parameters. Ways of determining T_{geffect} and the parameters are disclosed in Properties of Polymers by D. W. van Krevelen, Klaas te Nijenhuis, 4^{th} edition, Elsevier Science, 2009.

Other suitable equations for the matrix viscosity for various polymer reaction systems are provided in Properties of Polymers by D. W. van Krevelen, Klaas te Nijenhuis, 4^{th} edition, Elsevier Science, 2009.

In an aspect the morphology functional may be provided in the form of

$$Y \left(\text{ψ,} {t}_{\text{F} ,} {t}_{\text{O}}\right) = {\int }_{{t}_{\text{F}}}^{{t}_{\text{O}}} \text{ψ} d t \qquad \text{(10)}$$

where t_{F} and t_{O} are two time instants, such that a t_{O} > t_{F} ≥ 0. Typically, t_{F} corresponds to the time instant of polymer cluster formation and t_{O} is a subsequent observation time instant. The value of Y increases with the cluster mobility function Ψ and with the difference (t_{O} - t_{F}) between the polymer cluster formation and observation instants.

For a chosen cluster mobility function Ψ, the morphology functional Y may be seen as a function of the polymer cluster formation time and the observation time, i.e. Y = Y_{Ψ}(t_{F}, t_{O}). In this case, Y = Y_{ψ}(t_{F},t_{O}) is a two-variable function, which can graphically be depicted as a surface.

Furthermore, for a chosen cluster mobility function Ψ and a chosen observation time t_{O}, the morphology functional Y may be seen as a function of the polymer cluster formation time, i.e. Y = Y_{Ψ,to}(t_{F}) with domain 0 ≤ t_{F} < t_{O}. In this case, Y = Y_{Ψ,to}(t_{F}) is a function of a single variable, which can graphically be depicted as a curve.

When the reaction progression variable is time, the value of the morphology functional may be evaluated using Equation (9) directly. When the reaction progression variable is not time, the corresponding variable transformation must be carried out, either before or after integration.

For instance, if the reaction progression is described in terms of the normalized time τ = t/t_{end}, the morphology functional may be evaluated using integration in τ

$$Y \left(\text{ψ,} {τ}_{\text{F} ,} {τ}_{\text{O}}\right) = {t}_{\text{end}} {\int }_{{τ}_{\text{F}}}^{{τ}_{\text{O}}} \text{ψ} d τ \qquad \text{(11)}$$

where τ_{F} and τ_{O} are the normalized times corresponding, respectively, to t_{F} and t_{O}. Alternatively, the morphology functional may also be evaluated using Equation (10) followed by a variable transformation t → τ(t).

For instance, if the reaction progression is described in terms of the overall monomer conversion Xₒᵥₑᵣₐₗₗ, the morphology functional may be evaluated using integration in Xₒᵥₑᵣₐₗₗ

$$Y \left(\text{ψ} , {X}_{\text{overall,F}} , {X}_{\text{overall,O}}\right) = {\int }_{{X}_{\text{overall,F}}}^{{X}_{\text{overall,O}}} \text{ψ} \left(\frac{d t}{d {X}_{\text{overall}}}\right) d {X}_{\text{overall}} \qquad \text{(12)}$$

where X_{overall,F} and X_{overall,O} are the overall monomer conversion values corresponding, respectively, to t_{F} and t_{O}. The term

$$\left(\frac{d t}{d {X}_{\text{overall}}}\right)$$

inside the integral is related to the rate of polymerization and so the following expression may also be used

$$Y \left(\text{ψ} , {X}_{\text{overall,F}} , {X}_{\text{overall,O}}\right) = {\int }_{{X}_{\text{overall,F}}}^{{X}_{\text{overall,O}}} \text{ψ} \left(\frac{{M}_{M}}{{r}_{p} V}\right) d {X}_{\text{overall}} \qquad \text{(13)}$$

where M_{M} is the amount of monomers added until the end of the polymerization, rₚ is the rate of monomer conversion to polymer, and V is the volume of the reaction mixture. This equation shows how the cluster mobility and the rate of polymerization act together to determine the particle morphology. Alternatively, the morphology functional may also be evaluated using Equation (10) followed by a variable transformation t → Xₒᵥₑᵣₐₗₗ(t).

The trajectory of the morphology functional up to an observation point along the reaction progression describes the movement of polymer clusters in multiphase latex polymer particles since the instance of the polymer cluster-formation up to the observation point for all polymer clusters created along the reaction progression of the reaction until the observation point.

If, without loss of generality, the reaction progression variable is the overall monomer conversion Xₒᵥₑᵣₐₗₗ, and the formation point is denoted by the subscript F and the observation point is denoted by the subscript O, the trajectory of the morphology functional is then the curve:

$$Y = {Y}_{\text{ψ} , {X}_{\text{overall,O}}} \left({X}_{\text{overall,F}}\right) , \text{with domain 0} \leq {X}_{\text{overall,F}} < {X}_{\text{overall,O}} \qquad \text{(14)}$$

Each point of the trajectory of the morphology function is calculated, as explained above, using equation (10), (12) or (13). If another reaction progression variable is chosen, the calculation procedure will be analogous.

In an aspect the step of providing to the processing device comprises providing time series data from a production process, suitable for determining
a quantity indicative of the polymer or monomer content inside the polymer matrix in the production reaction process
and a production reaction progression variable.

In a further aspect, the reaction progression trajectory of the morphology functional up to an observation point is determined based on
the reaction temperature
the glass transition temperature of the seed polymer.

In an aspect the reaction progression trajectory of the morphology functional up to an observation point is determined based on
the time series data from a reference polymerization process, suitable for determining:
   the quantity indicative of the polymer or monomer content inside the polymer particle in the reference polymerization process,
   and the reference reaction progress variable.

In an aspect the reference trajectory of the morphology functional may be determined by calculating

$${Y}_{\text{ψ}}^{\text{ref}} ,$$

using the provided time series data from a reference polymerization process, suitable for determining:
the quantity indicative of the amount of polymerized monomers in the reference polymerization process and
the quantity indicative of the polymer or monomer content inside the polymer particle in the reference polymerization process and the reference reaction progression variable.

The reference trajectory of the morphology functional up to an observation point along the reaction progression describes the movement of polymer clusters in multiphase latex polymer particles since the instance of the polymer cluster-formation up to the observation point for all polymer clusters created along the reaction progression of the reaction until the observation point.

In an aspect this observation point may be a point in time.

In another aspect the observation point may be a specific overall monomer conversion. The overall monomer conversion is a measure of the progress of conversion of monomers and therefore a measure for the reaction progression. The specific overall monomer conversion may be any point along the progression of the reaction.

Determining the reference trajectory of the morphology functional is in particular useful to identify reaction processes that provide similar morphology.

Similarity in morphology can be achieved when the reference reaction process shows similarity to the production process. Similarity can be considered when the cluster mobility functions are similar. This may be the case when the matrix viscosity is substantially the same. The cluster mobility function is closely related to the glass transition temperature of the polymer seed. The cluster mobility function may also be closely related to the molar mass of the polymer seed. Similar driving forces are expected when polymers of similar hydrophobicity and similar surfactants are used. This is maintained when the chemical composition is similar. Furthermore, similarity requires the same seed size and the same seed/second stage polymer ratio. For identical formulations similarity is trivial. The position in the polymer particle where the polymer clusters are formed depends on the type of initiator used (water-soluble vs. oil-soluble) and no significant differences in similarity occur during the controlled polymerization if the same initiator is used.

The reference reaction may be a reaction at a second stage of the synthesis of heterogeneous latex particles in a semi-batch operation.

In an aspect the reference morphology functional describes the movement of polymer clusters in multiphase latex polymer particles since the instance of the polymer cluster formation along up to the observation point for all polymer clusters created along the reaction progression of the reference reaction. In some cases the observation point may be the endpoint of the reference reaction.

In an aspect the reference trajectory of the morphology functional is determined as a setpoint value for controlling.

In an aspect determining at the processing device comprises determining a production trajectory of the morphology functional up to the recent observation point along the production reaction progression based on
the time series data from the production process, suitable for determining,
   the quantity indicative of the polymer or monomer content inside the polymer particle in the production process
   and the production reaction progression variable.

The recent observation point may also refer to a current observation point.

In an aspect the production trajectory of the morphology functional may be determined by calculating

$${Y}_{\text{ψ}}^{\text{prod}} ,$$

using the provided
time series data from the production process, suitable for determining,
   the reaction progression variable, and
   the quantity indicative of a polymer and/or monomer content inside the polymer particle in the reference polymerization process.

In an aspect the reference and production trajectory of the morphology functional may be determined using the same morphology functional and reaction progression variable.

In an aspect the production trajectory of the morphology functional is determined as an actual value input signal for controlling.

For controlling, two values need to be provided to a control algorithm. The setpoint value and the actual value input signal. In the controller these signals are evaluated to determine the accurate action such that the deviation from the setpoint value is minimized.

In an aspect the setpoint value and the actual value input are provided to a control algorithm. The control algorithm may be located in the same processing device or in a distinct processing device. Providing may include providing via an interface.

In the following section two out of many suitable algorithms for controlling are described in more detail.

A next step may comprise receiving the setpoint value and the actual value input at the control algorithm.

The control algorithm may determine the control actions so that the production trajectory of the morphology functional matches the reference trajectory of the morphology functional.

A first algorithm is described based on a recent situation of the production process with respect to the reference polymerization process expressed in terms of a morphology trajectory.

In an aspect the control algorithm comprises a step of comparing the production trajectory of the morphology functional with the reference trajectory of the morphology functional up to the respective observation point along the reaction progression. In this type of algorithm the reference observation point and the recent observation point refer to the same point in the reaction progression.

The control algorithm may comprise a step of comparing the setpoint value to the actual value input signal.

By this for all polymer clusters formed in the production process their movement is compared to the movement that the polymer clusters formed at a similar point along the reaction progression of the reference polymerization process have undergone.

In an aspect comparing the production trajectory of the morphology functional

$$\left({Y}_{\text{ψ}}^{\text{prod}}\right)$$

with the reference trajectory of the morphology functional

$$\left({Y}_{\text{ψ}}^{\text{ref}}\right)$$

comprises the step of determining the difference between the production trajectory of the morphology functional and the reference trajectory of the morphology functional up to the observation point along the reaction progression.

In an aspect the result of the comparison is an error calculated as

$$\begin{matrix}e = {\int }_{0}^{X {}_{\text{overall,O}}} W \left(X {}_{\text{overall,F}}\right) \\ {\left({Y}_{\text{ψ,} X {}_{\text{overall,O}}}^{\text{prod}} \left(X {}_{\text{overall,F}}\right) - {Y}_{\text{ψ,} X {}_{\text{overall,O}}}^{\text{ref}} \left({X}_{\text{overall,F}}\right)\right)}^{2} d {X}_{\text{overall,F}}\end{matrix} \qquad \text{(15)}$$

wherein W(X_{overall,F}) is a weighing function that may be used to give more importance to certain parts of the trajectory. In an aspect, W(X_{overall,F}) = 1, for all X_{overall,F},

In an aspect the method comprises a step of minimizing the error signal.

As previously described, X_{overall,F}, and

$${Y}_{\text{ψ,} X {}_{\text{overall,O}}}^{\text{prod}} ,$$

may be derived from the reaction temperature, the flowrates of each ingredient into the reactor, the initial amount of monomers and the quantity indicative of a polymer and/or monomer content inside the polymer matrix in the reaction.

In an aspect the step of minimizing the error signal may comprise manipulating the monomer feed rate and/or manipulating the initiator feed.

In an aspect manipulating the monomer feed may be increasing the monomer feed and manipulating the initiator feed may be decreasing the initiator feed. This is in particular useful when the step of comparing the determined trajectory with the reference trajectory of the morphology functional up to the observation point along the reaction progression reveals that the polymer clusters in the production process are moving slower than polymer clusters formed at the same point along the reaction progression in the reference polymerization process. By that the monomer concentration increases which reduces the viscosity and thereby increases cluster mobility without accelerating the conversion of monomer to polymer. This may be advantageous if a tight control regime with fast correction of disturbances is required. Feed control is generally faster than temperature control.

In an aspect manipulating the monomer feed may be decreasing the monomer feed and manipulating the initiator feed may be increasing the initiator feed. This is in particular useful when the step of comparing the determined trajectory with the section of the reference trajectory of the morphology functional up to the observation point along the reaction progression reveals that the polymer clusters in the production process are moving faster than similar polymer clusters in the reference polymerization process. By that the monomer concentration decreases which increases the viscosity without slowing the polymerization process. This may be advantageous if a tight control regime with fast reaction to disturbances is required. Feed control is generally faster than temperature control.

In an aspect the step of minimizing the error signal may comprise manipulating the reaction temperature.

In an aspect the reaction temperature may be increased. This is in particular useful when the step of comparing the determined trajectory with the section of the reference trajectory of the morphology functional up to the observation point along the reaction progression reveals that the polymer clusters in the production process are moving slower than similar polymer clusters in the reference polymerization process. Increasing the reaction temperature in the reaction process increases the movement of the polymer clusters in the production process this may be complemented by a decrease of the amount of initiator to compensate for the effect of temperature on polymerization rate. This may be useful for small reactors having a low heat capacity.

In an aspect the reaction temperature may be decreased. This is in particular useful when the step of comparing the determined trajectory with the section of the reference trajectory of the morphology functional up to the observation point along the reaction progression reveals that the polymer clusters in the production process are moving faster than similar polymer clusters in the reference polymerization process. Decreasing the reaction temperature in the reaction process decreases the movement of the polymer clusters in the production process this may be complemented by an increase of the amount of initiator to compensate for the effect of temperature on polymerization rate. This may be useful for small reactors having a low heat capacity.

A second algorithm is described below.

The second algorithm is based on using the morphology functional in a model predictive controller.

The algorithm may comprise computing optimal changes in manipulated variables (e.g. flow rates of each ingredient into the reactor, temperature of the reaction, temperature of the ingredients flowing into the reactor) based
on the production trajectory of the morphology functional at the recent observation point
the morphology functional and
the reference observation point,
such that the deviation between the predicted product trajectory and the reference trajectory of the morphology functional at the reference observation point is minimized.

In an aspect the reference observation point represents a reaction progression further advanced than reaction progression at the recent observation point. In terms of time and/or overall monomer conversion it may be considered as a future observation point in the recent observation point. The difference in progression of the reaction between the recent observation point and the reference observation point may be described as prediction horizon.

In an aspect the prediction horizon may be provided.

In an aspect the algorithm comprises minimizing the difference between the predicted production trajectory of the morphology functional and the reference trajectory of the morphology functional at the reference observation point.

Minimizing can be achieved by calculating control actions (e.g., evolution of the flow rates on monomers, initiator, and cooling/heating fluid and the evolution of inlet temperature of the cooling/heating fluid), until the observation point in the prediction horizon.

In an aspect the evolution of the flow rates of monomers, initiator, and cooling/heating fluid and the evolution of inlet temperature of the cooling/heating fluid, collectively represented as a vector of manipulated variables u(t), may be determined as

$$\begin{matrix}{\text{min}}_{u \left(t\right)} \left[{\int }_{0}^{X {}_{\text{overall,PH}}} W \left(X {}_{\text{overall,F}}\right)\right) \\ \left({\left({Y}_{\text{ψ,} X {}_{\text{overall,PH}}}^{\text{prod}} \left(X {}_{\text{overall,F}}\right) - {Y}_{\text{ψ,} X {}_{\text{overall,PH}}}^{\text{ref}} \left({X}_{\text{overall,F}}\right)\right)}^{2} d {X}_{\text{overall,F}}\right]\end{matrix} \qquad \text{(15)}$$

wherein X_{overall,PH} is the overall monomer at the observation point in the prediction horizon and equation (15) is subjected to material and heat balances in the reactor.

In equation (15), W(X_{overall,F}) is a weighing function that may be used to give more importance to certain parts of the trajectory. In an aspect, W(X_{overall,F}) = 1, for all X_{overall,F},

Minimizing may be achieved by calculating production reaction conditions to the reference point such that the predicted product trajectory matches the reference trajectory of the morphology functional at the reference observation point.

In an aspect the future reaction conditions expressed as the time evolutions of the amount of unreacted monomers (or equivalently the instantaneous monomer conversion Xᵢₙₛₜ), overall monomer conversion and temperature may be determined as

$$\begin{matrix}{\text{min}}_{{X}_{\text{inst}} \left(t\right) , X {}_{\text{overall}} \left(t\right) , T \left(t\right)} \left[{\int }_{0}^{X {}_{\text{overall,PH}}} W \left(X {}_{\text{overall,F}}\right)\right) \\ \left({\left({Y}_{\text{ψ,} X {}_{\text{overall,PH}}}^{\text{prod}} \left(X {}_{\text{overall,F}}\right) - {Y}_{\text{ψ,} X {}_{\text{overall,PH}}}^{\text{ref}} \left({X}_{\text{overall,F}}\right)\right)}^{2} d {X}_{\text{overall,F}}\right]\end{matrix} \qquad \text{(16)}$$

wherein W(X_{overall,F}) is a weighing function that may be used to give more importance to certain parts of the trajectory. In an aspect, W(X_{overall,F}) = 1, for all X_{overall,F},

In an aspect the point of observation in the future may be the end of the reaction process.

In an aspect Xᵢₙₛₜ(t), Xₒᵥₑᵣₐₗₗ (t), T(t) define the process that leads to the sought particle morphology. This presents the advantage that they are variables that can be easily monitored as described above. These variables depend on flowrate of each of the ingredients, temperature of each of the ingredients and the temperature of the cooling/heating jacket. Which are available throughout the reaction process as directly measured variables. The control algorithm may therefore provide set points for each of these directly measured variables.

Many ways of feedback control may be used, e.g. proportional-integral-derivative controller (PID), model predictive controller (MPC), in particular non-linear model predictive controllers may be used for improved results.

In an aspect Xᵢₙₛₜ(t), Xₒᵥₑᵣₐₗₗ (t), T(t) are determined from the beginning of the semicontinuous operation.

In an aspect Xᵢₙₛₜ(t), Xₒᵥₑᵣₐₗₗ(t), T(t) are controlled by varying the feed rates of the ingredients of the formulation (monomers, initiators).

In an aspect Xᵢₙₛₜ(t), Xₒᵥₑᵣₐₗₗ (t), T(t)are controlled by modifying the inlet temperature and/or the flow rate of the cooling/heating fluid.

In an aspect in the absence of perturbations and substantial changes in the reactor type and size Xᵢₙₛₜ(t), Xₒᵥₑᵣₐₗₗ(t), T(t) can be those of the reference polymerization process.

The method has the advantage that the morphology of polymer particles in emulsion polymerization in particular in a second or later stage of the emulsion polymerization in a reactor may be described by a simple equation of movement of polymer clusters in the particle. Up to now controlling of morphology required for each polymer system a full model including reaction kinetics and morphology dynamics. In practice, this implies the development of a model for each system, which can be acceptable for some large tonnage products, but is not convenient in a market that is characterized by large portfolios of medium or low tonnage products.

In an aspect the method is for an emulsion polymerization in which the morphology is undergoing unstable/non equilibrium morphologies. The proposed method is in particular useful for emulsion polymerizations, in which the morphology is undergoing unstable/non equilibrium morphologies. In addition, unstable morphologies are not easy to be produced in robust processes.

In another example the morphology functional may be used for optimizing production capacity while maintaining the morphology of polymer particles. The reference reaction process may be performed at low reaction speeds and in the production reaction process the reaction speed may be faster, however as long as the reaction progression follows the trajectory of the morphology functional of the reference reaction process, the morphology will be reproduced. This may reduce the number of experiments needed to proceed to optimal production speeds. Additionally, due to fluctuations in the production process from batch to batch, this will result in optimum reaction time for each and every batch.

In an aspect the step of providing a reference trajectory of the morphology functional comprises providing the reference trajectory of the morphology functional as a setpoint value to a control algorithm.

In an aspect providing to the processor comprises providing production constraints.

Production constraints are constraints of a production reactor system unit.

The production reactor system unit comprises a reactor, and may comprise ingredient feeds, a temperature sensor for each of the ingredient feeds, a cooling/heating jacket, a reactor volume.

In an aspect the step of minimizing includes optimizing the production reaction time while reproducing the reference polymer particle morphology under consideration of the production constraints.

In an aspect the step of minimizing comprises a multi-criterium optimization

$$\begin{matrix}{\text{min}}_{u \left(t\right)} \left[\left({\int }_{0}^{X {}_{\text{overall,PH}}} W \left(X {}_{\text{overall,F}}\right)\right)\right) \left({Y}_{\text{ψ,} X {}_{\text{overall,PH}}}^{\text{prod}} \left(X {}_{\text{overall,F}}\right) -\right) \\ \left(\left({\left({Y}_{\text{ψ,} X {}_{\text{overall,PH}}}^{\text{ref}} \left({X}_{\text{overall,F}}\right)\right)}^{2} d {X}_{\text{overall,F}}\right) + {\sum }_{i} {w}_{i} {P}_{i}\right]\end{matrix} \qquad \text{(16)}$$

where Pᵢ are penalty functions and wᵢ are the corresponding weights, and equation (16) is subjected to material and heat balances in the reactor and the production constraints. The penalty functions may comprise productivity loss functions, which set a penalty when the capacity of the production system is not fully utilized. The production constraints comprise at least one of the list of:
Reactor volume,
Heat removal capacity of the reactor (maximum cooling fluid flow rate and minimum inlet temperature),
Reaction temperature range (maximum and minimum),
Cooling fluid temperature range,
Cooling fluid flow rate range,
Amount of monomers to be fed,
Maximum feed rate for each ingredient,
Maximum variation of the feed rates (this refers to the limitations in the pumps to make sudden changes in flow rates),
Maximum amount of unreacted monomers in the reactor (safety reasons in case of failure of the cooling system. Also to avoid formation of monomer droplets)

According to an aspect, a computer program or a computer program product or computer readable non-volatile storage medium comprising computer readable instructions, which when loaded and executed by a processing device perform the methods disclosed herein.

According to an aspect a system is proposed, the system comprising an input device, and output device and a processing device configured for performing the method disclosed herein.

The processing device may be a computer or even a general-purpose processing device such as a microprocessor, microcontroller, central processing unit (“CPU”), or the like. More particularly, the processing device may be a CISC (Complex Instruction Set Computing) microprocessor, RISC (Reduced Instruction Set Computing) microprocessor, VLIW (Very Long Instruction Word) microprocessor, or a processor implementing other instruction sets or processors implementing a combination of instruction sets. The processing device or processing means may also be one or more special-purpose processing devices such as an ASIC (Application-Specific Integrated Circuit), an FPGA (Field Programmable Gate Array), a CPLD (Complex Programmable Logic Device), a DSP (Digital Signal Processor), a network processor, or the like. The methods, systems and devices described herein may be implemented as software in a DSP, in a micro-controller, or in any other side-processor or as hardware circuit within an ASIC, CPLD, or FPGA. As outlined also earlier, it is to be understood that the term “processing device” or processor may also refer to one or more processing devices, such as a distributed system of processing devices located across multiple computer systems (e.g., cloud computing), and is not limited to a single device unless otherwise specified. Moreover, any one or more of the processing devices may be located at a physical location which is different from the other processing devices.

The disclosure applies to the systems, methods, computer programs, computer readable non-volatile storage media, computer program products disclosed herein alike. Therefore, no differentiation is made between systems, methods, computer programs, computer readable non-volatile storage media or computer program products. All features are disclosed in connection with the systems, methods, computer programs, computer readable non-volatile storage media, and computer program products disclosed herein.

The interface for providing to the processor may comprise a physical interface (e.g. a keyboard, a touch screen, a computer mouse, etc.) The interface for providing to the processor may comprise a logical interface (e.g. computer interface to a database, a wired or wireless interface to a computer or a computer network, etc.). In another aspect the interface for providing to the processor may be one interface or several interfaces. In particular, each determination step may be performed at a separate processor, implying that for each providing step a separate interface may be used.

### DESCRIPTION OF THE FIGURES

FIG. 1: illustrates a production reactor setup suitable for use in emulsion polymerization reactions.

FIG. 2 illustrates a reference reactor setup suitable for use in emulsion polymerization reactions.

FIG. 3 illustrates movement of a polymer cluster inside a particle during progression of a reaction.

FIG. 4 illustrates the workflow of the proposed method.

FIG. 5 illustrates the workflow of a suitable control algorithm

FIG. 6 illustrates a setup comprising the system for performing the control method and the connection to a setup for performing the reaction.

FIG. 7illustrates trajectories according to the disclosure

FIG. 8 illustrates movement of polymer clusters along the reaction progression

FIG. 9 illustrates a soft sensor according to the invention

FIG. 10 shows a distributed system for providing a soft sensor according to the invention

FIG. 11 shows an alternative setup for controlling

FIG. 12 shows a method of producing a soft sensor according to the invention

FIG. 13 shows a system for producing a soft sensor according to the invention

### DETAILED DESCRIPTION

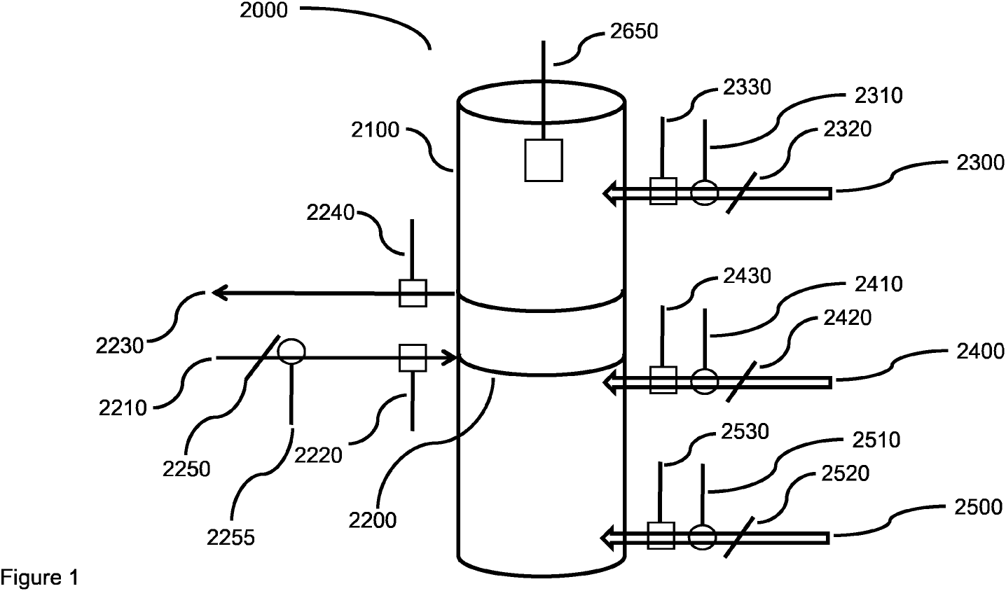

In FIG. 1 a production reactor setup 2000 suitable for use in emulsion polymerization reactions is depicted. A reactor 2100 is equipped with a cooling/heating jacket 2200, for heating and/or cooling the reactor. The cooling/heating jacket does have an inlet 2210 and an outlet 2230. The inlet of the cooling/heating jacket is provided with an inlet temperature sensor 2220, that measures the inlet temperature (T_{jacket,in}). The outlet of the cooling/heating jacket is provided with an outlet temperature sensor 2240, that measures the outlet temperature (T_{jacket,out}). The inlet of the cooling/heating jacket is equipped with an actuator 2250 for manipulating the flowrate of the cooling medium in the cooling/heating jacket and a flow sensor 2255 for measuring that flowrate. In another example not shown, there can be more than one cooling/heating jackets. In further examples cooling and heating may be performed by one or more separate devices. The reactor is further equipped with a temperature sensor 2650 for measuring the reaction temperature in the reactor. The reactor is equipped with three ingredient feedlines 2300, 2400, 2500. In this example feedline 2300 is for feeding a first monomer, feedline 2400 is for feeding a second monomer and feedline 2500 is for feeding an initiator. Each of the feedlines is provided with a respective flow sensor 2310, 2410 and 2510 a respective temperature sensor 2330, 2430, 2530 and a respective actuator 2320, 2420, 2520 for manipulating the respective flowrate. The first to digits of the flow sensors, the temperature sensors and the actuators refer to the corresponding feedline.

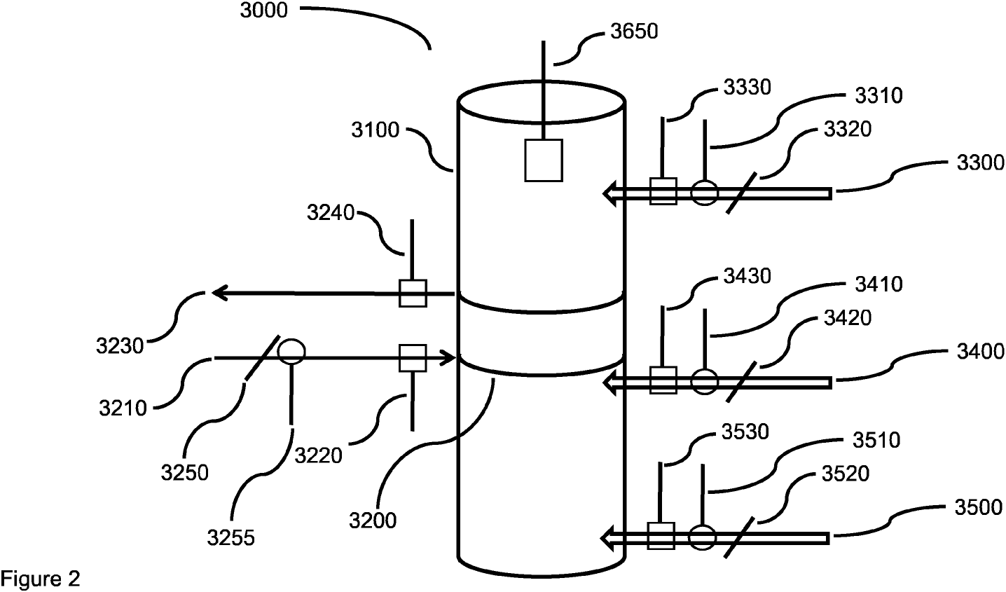

In FIG. 2 a reference reactor system 3000 similar the reactor system in FIG. 1 is shown. Numbers start at 3000 and items corresponding to items in FIG. 1 use the same last three digits as in FIG. 1.

The reactor is further equipped with a temperature sensor 3650 for measuring the reaction temperature in the reactor. The reactor is equipped with three ingredient feedlines 3300, 3400, 3500. In this example feedline 3300 is for feeding a first monomer, feedline 3400 is for feeding a second monomer and feedline 3500 is for feeding an initiator. Each of the feedlines is provided with a respective flow sensor 3310, 3410 and 3510 a respective temperature sensor 3330, 3430, 3530 and a respective actuator 3320, 3420, 3520 for manipulating the respective flowrate. The first to digits of the flow sensors, the temperature sensors and the actuators refer to the corresponding feedline.

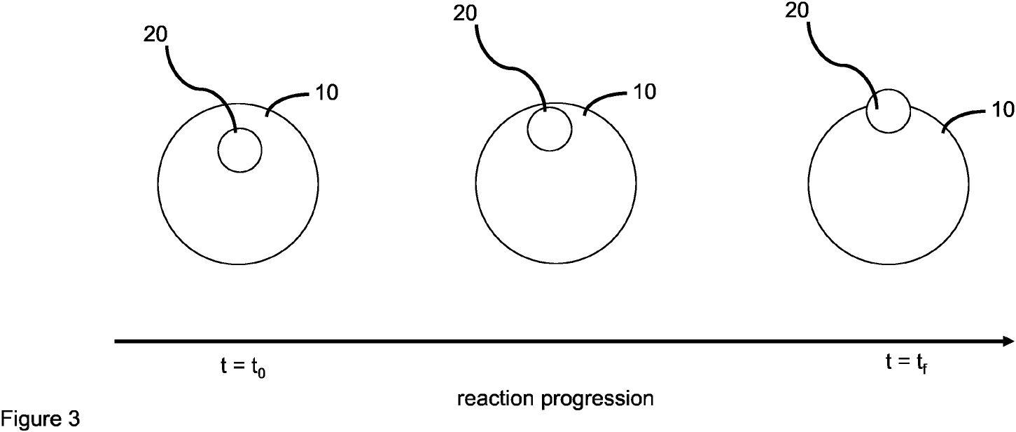

FIG. 3 illustrates how the morphology of a polymer particle 10 depends on movement of polymer clusters. A polymer cluster 20 formed at a time t=0 will in this illustration migrate to a final position at the end of the polymer reaction t=t_{f}. The final position of the polymer cluster would lead to a raspberry like structure of the polymer particle. For illustration purposes only one polymer cluster is formed at t=0 although typically a larger number of polymer clusters may be formed at each time during the reaction process. Therefore, when discussing a polymer cluster formed at a specific point in time it is generally referred to all polymer clusters formed at that specific time and the movement may be a mean movement considering all polymer clusters formed at that specific time. FIG. 3 also neglects that new polymer clusters may be formed during any point of the reaction progression.

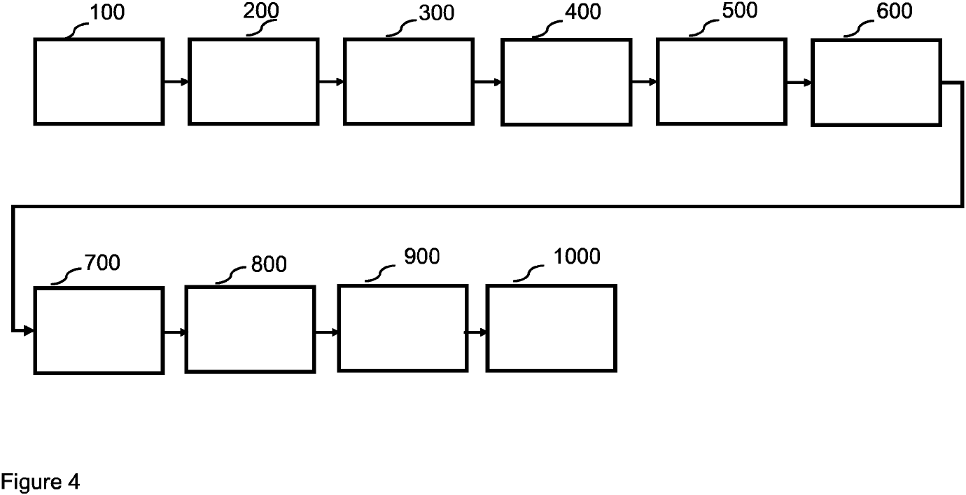

In FIG. 4 the method of controlling is illustrated in a non-limiting example.

In this example a first monomer and a second monomer are used. An initiator is also used. The selection of monomer and initiator is for illustration purposes only and should not be construed as limiting. The method is applicable for other combinations of monomers and initiators alike.

At step 100 time series data from a reference polymerization process, suitable for determining
the reaction progression variable
a quantity indicative of the polymer or monomer content inside the polymer particles in the reference polymerization process,
is provided to processor 4200.

The time series data from the reference polymerization process comprises the temperature of the reactor. In this example the reaction temperature (T) in the reactor is measured by the temperature sensor 3250 in the reactor 3100;
The cooling/heating jacket temperature; the inlet and outlet temperatures are measured in this example by the temperature sensors 3220 and 3240 respectively;
The flowrates of each ingredient fed into the reactor comprises measured data from the flow sensors for each of the type of monomers 3310, 3410 and the initiator 3510 throughout the entire course of the polymerization: ∀ᵢ, Fᵢ(t), 0 ≤ t ≤ t_{end}. Wherein Fi is the flow rate for each ingredient and i is an index for each type of ingredient; un this example the indices are assigned as follows (i= 1: first monomer; i=2: second monomer; i=3: initiator);
The temperature of the feeds measured by temperature sensors 3330, 3430 and 3530;
the initial amount of each ingredient in the reactor comprises the initial amount of a first monomer, an initial amount a second monomer and an initial amount of the initiator;
the flowrate of the cooling medium in the cooling/heating jacket measured by flow sensor 3255.

From these time series data the quantity indicative of the amount of polymerized monomers in the reaction process is determined at optional step 200.

The amount of heat produced by polymerization (Q) is then determined by means of the heat balance in the reactor using equation (2)

The mass flow of each ingredient is provided to the processor from the flow sensors 2310, 2410, 2510.

The amount of monomer converted to polymer may then be determined as

$$\left\{M o l e s o f m o n o m e r p o l y m e r i z e d\right\} = \frac{1}{\text{Δ} {Η}_{p o l}} {\int }_{0}^{t} \dot{Q} d {t}^{′}$$

Wherein ΔHₚₒₗ is the enthalpy of the polymerization reaction. If more than one type of monomers is used, data indicative of the amount of each monomer i polymerized (M_{i,polymerized}) and the amount of polymer can be obtained by using a modification of equation (3) as described in literature[Gugliotta, L.M., Arotçarena M., Leiza, J.R., Asua, J.M. Estimation of conversion and copolymer composition in semi-continuous emulsion polymerization using calorimetric data, Polymer, 1995, 36, 2019-2013].

At optional step 300 the quantity indicative of the polymer or monomer content inside the polymer matrix in the reference reaction process is determined. In this example it is derived from the quantity indicative of the amount of monomer converted to polymer. In this example the assumption that the whole amount of monomer converted to polymer is inside the particles and that the remaining amount of unreacted monomer is also inside the particles is used. In this example, the volume fraction of polymer in the polymer particles can then be determined using the relation

$$\begin{matrix}{\text{ϕ}}_{p o l} \left(t\right) \approx \\ \frac{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}}}{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}} + \frac{\left\{M a s s o f u n r e a c t e d m o n o m e r s\right\}}{\left\{D e n s i t y o f m o n o m e r s\right\}}}\end{matrix}$$

In this example it is assumed to be the quantity indicative of the polymer content inside the polymer matrix.

Step 400 is again optional, here time series data from a production process, suitable for determining
a reaction progression parameter and
a quantity indicative of the polymer or monomer content inside the polymer particles in the production reaction process, are provided to the processor.

The time series data from the reference polymerization process comprises:
the temperature of the reactor 3100. In this example the reaction temperature (T) in the reactor is measured by the temperature sensor 3250 in the reactor 3100;
The cooling/heating jacket temperature; the inlet and outlet temperatures are measured in this example by the temperature sensors 3220 and 3240 respectively;
The flowrates of each ingredient fed into the reactor comprises measured data from the flow sensors for each of the type of monomers 3310, 3410 and the initiator 3510 throughout the entire course of the polymerization: ∀ᵢ, Fᵢ(t), 0 ≤ t ≤ t_{end}. Wherein Fi is the flow rate for each ingredient and i is an index for each type of ingredient; un this example the indices are assigned as follows (i= 1: first monomer; i=2: second monomer; i=3: initiator);
The temperature of the feeds measured by temperature sensors 3330, 3430 and 3530;
the initial amount of each ingredient in the reactor comprises the initial amount of a first monomer, an initial amount a second monomer and an initial amount of the initiator;
the flowrate of the cooling medium in the cooling/heating jacket measured by flow sensor 3255.

From these time series data the quantity indicative of the amount of polymerized monomers in the reaction process is determined at optional step 500.

The amount of heat produced by polymerization (Q) is then determined by means of the heat balance in the reactor using equation (2)

$${m}_{R} {c}_{p R} \frac{d T}{d t} = \dot{Q} + {\sum }_{i} {F}_{i} {c}_{p i} \left({T}_{i} - T\right) - {\dot{Q}}_{j}$$

Wherein m_{R} and c_{pR} are the mass and the specific heat capacity of the reactor and Fᵢ and cₚᵢ the mass flow and specific heat capacity of ingredient i that is fed to the reactor at a temperature Tᵢ. The mass flow of each ingredient is provided to the processor from the flow sensors 3310, 3410, 3510.

The amount of monomer converted to polymer may then be determined as

$$\left\{M o l e s o f m o n o m e r p o l y m e r i z e d\right\} = \frac{1}{\text{Δ} {Η}_{p o l}} {\int }_{0}^{t} \dot{Q} d {t}^{′}$$

Wherein ΔHₚₒₗ is the enthalpy of the polymerization reaction. If more than one type of monomers is used, data indicative of the amount of each monomer i polymerized (M_{i,polymerized}) and the amount of polymer can be obtained by using a modification of equation (3) as described in literature [Gugliotta, L.M., Arotçarena M., Leiza, J.R., Asua, J.M. Estimation of conversion and copolymer composition in semicontinuous emulsion polymerization using calorimetric data, Polymer, 1995, 36, 2019-2013].

At optional step 600 the quantity indicative of the polymer or monomer content inside the polymer particles in the production reaction process is determined. In this example it is derived from the quantity indicative of the amount of monomer converted to polymer. In this example the assumption that the whole amount of monomer converted to polymer is inside the particles and that the remaining amount of unreacted monomer is also inside the particles is used. In this example, the volume fraction of polymer in the polymer particles can then be determined using the relation

$$\begin{matrix}{\text{ϕ}}_{p o l} \left(t\right) \approx \\ \frac{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}}}{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}} + \frac{\left\{M a s s o f u n r e a c t e d m o n o m e r s\right\}}{\left\{D e n s i t y o f m o n o m e r s\right\}}}\end{matrix}$$

In this example it is assumed to be the quantity indicative of the polymer content inside the polymer matrix.

At step 700 the morphology functional is provided to the processor, wherein the morphology functional maps the morphology functional describes the movement of polymer clusters in multiphase latex polymer particles since the instance of the polymer cluster-formation along a reaction progression in a reaction process, the morphology functional depending on the time series data, suitable for determining a quantity indicative of the amount of polymerized monomers in the reaction process, a quantity indicative of the polymer or monomer content inside the polymer particles in the reaction process and a reaction progression variable in this example the reaction progression variable is the overall monomer Xₒᵥₑᵣₐₗₗ.

In this example the morphology functional depends on the conditions inside the polymer particle. This dependency is in this example described by the cluster mobility function (Ψ). Therefore, in this example the morphology functional relationship comprises the cluster mobility function. The cluster mobility function describes the mobility in the polymer particles during progression of the reaction process.

In an example the cluster mobility function depends on the data indicative of the reaction temperature, the quantity indicative of monomer or polymer content inside the polymer matrix and the glass transition temperature (T_{g}) and the molar mass (M_{w}) of the seed polymer. In this example the cluster mobility function is considered to be the matrix viscosity (η).

$$\text{ψ} = \frac{1}{η \left({ϕ}_{p o l} , T , {T}_{g} , {M}_{w}\right)}$$

The morphology functional (Y) in this example is a functional that depends on the cluster mobility function (Ψ) and Xₒᵥₑᵣₐₗₗ; Y=f(Ψ,Xₒᵥₑᵣₐₗₗ).

In this example the, the particle matrix viscosity is described as:

$$\left(η \left({ϕ}_{p o l} , T , {T}_{g} , {M}_{w}\right) = A {ϕ}_{p o l}^{n} \text{exp} \left(B \frac{{Τ}_{g , e f f}}{T} - C\right)\right) \qquad \text{(9)}$$

Wherein T_{g,eff} is the effective glass transition temperature, and A, n, B and C are parameters that can be experimentally determined. Ways of determining T_{g,eff} and the parameters are disclosed in Properties of Polymers by D. W. van Krevelen, Klaas te Nijenhuis, 4^{th} edition, Elsevier Science, 2009. In this example, the effect of the molar mass of the seed is included in the parameters. In an alternative example a relative matrix viscosity is used (ηᵣₑₗ).

At optional step 800 as an actual value input signal the production trajectory of the morphology functional up to a recent observation point along the production reaction progression based on the time series data from a production process, and the production reaction progression variable is determined.

At step 900 as a setpoint value the reference trajectory of the morphology functional up to a reference observation point along the production reaction progression based on
the time series data from the reference polymerization process,
and the reference reaction progression variable is determined.

At optional step 1000, the setpoint value and the actual value input signal a provided to a control algorithm, wherein the control algorithm determines a value for manipulated variables based on the setpoint value and the actual value input signal.

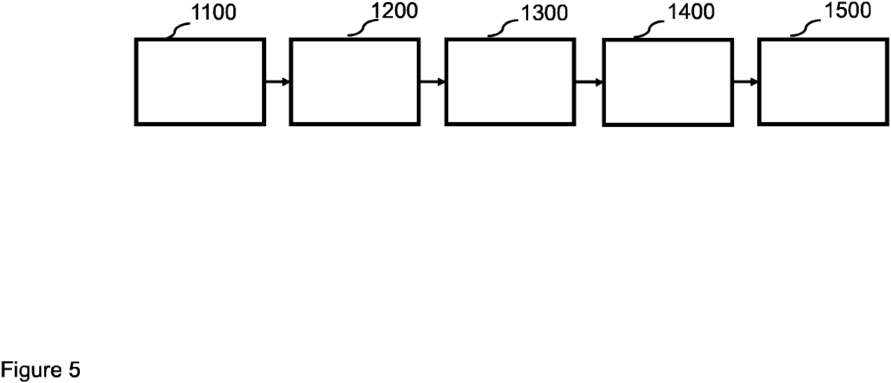

The optional steps of a suitable control algorithm are shown in FIG. 5.

At optional step 1100, the setpoint value and the actual value input signal are received by the control algorithm.

At step 1200 a predicted production trajectory of the morphology functional is determined based on the production trajectory of the morphology functional at the recent observation point, the morphology functional and the reference observation point.

In step 1300 the future reaction conditions expressed as the time evolutions of the amount of unreacted monomers (or equivalently the instantaneous monomer conversion Xᵢₙₛₜ), overall monomer conversion and temperature may be determined as

$$\begin{matrix}{\text{min}}_{{X}_{\text{inst}} \left(t\right) , X {}_{\text{overall}} \left(t\right) , T \left(t\right)} \left[{\int }_{0}^{X {}_{\text{overall,PH}}} W \left(X {}_{\text{overall,F}}\right)\right) \\ \left({\left({Y}_{\text{ψ,} X {}_{\text{overall,PH}}}^{\text{prod}} \left(X {}_{\text{overall,F}}\right) - {Y}_{\text{ψ,} X {}_{\text{overall,PH}}}^{\text{ref}} \left({X}_{\text{overall,F}}\right)\right)}^{2} d {X}_{\text{overall,F}}\right]\end{matrix}$$

wherein W is a weighing function that may be used to give more importance to certain parts of the trajectory. In an aspect, W(X_{overall,F}) = 1, for all X_{overall,F},

This determines the production trajectory of the morphology functional with the least deviation from the reference trajectory of the morphology functional. In terms of control, the difference between the recent observation point and the reference observation point is the prediction horizon.

At step 1400, Xᵢₙₛₜ(t), Xₒᵥₑᵣₐₗₗ(t), T(t) are controlled by varying the feed rates of the ingredients of the formulation (monomers, initiators), and the inlet temperature and/or the flow rate of the cooling/heating fluid.

At step 1500 control signals are provided to manipulate the flow rates and the temperatures accordingly.

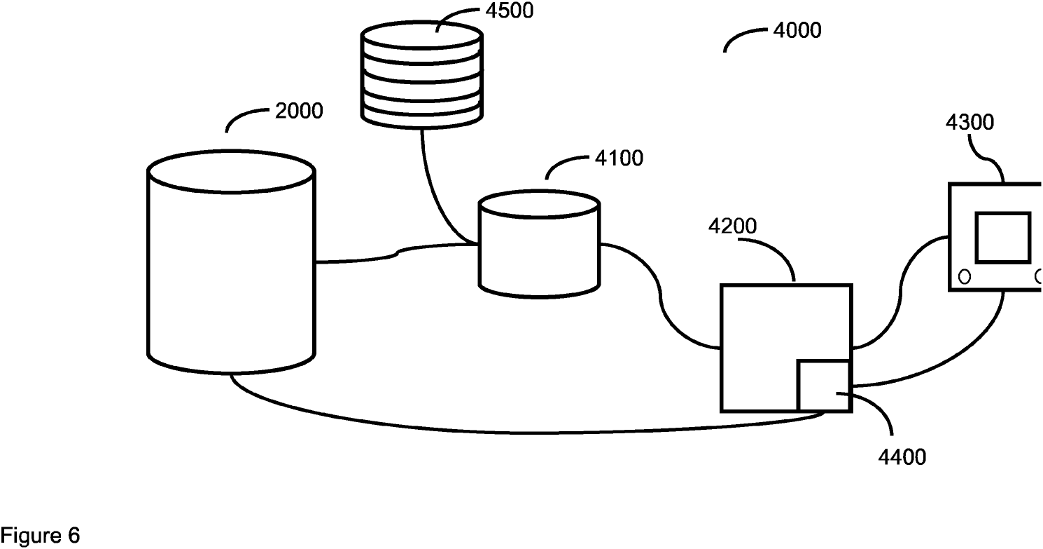

FIG. 6 is an illustration for a suitable system 4000.

The system comprises the processor 4200 to which data is provided. An interface 4100 receives the morphology functional from a database 4500. The time series data from the reference polymerization process are also provided from the database. The time series data for the reference polymerization process, the morphology functional provided to the processor via the interface 4100.

The time series data from the production process are provided from the production process system 2000 to an interface. In this example interface 4100. In other examples various different interfaces may be used. The time series data from the production process are provided to the processor via the interface.

The processor in this example also comprises the control algorithm 4400 and is located in the processor. In other examples the control algorithm may be hosted in a separate device.

The control algorithm provides control signals to the production system. 4300 depicts an input/output device for monitoring and inputting data. The prediction horizon may in an example be provided by the input/output device.

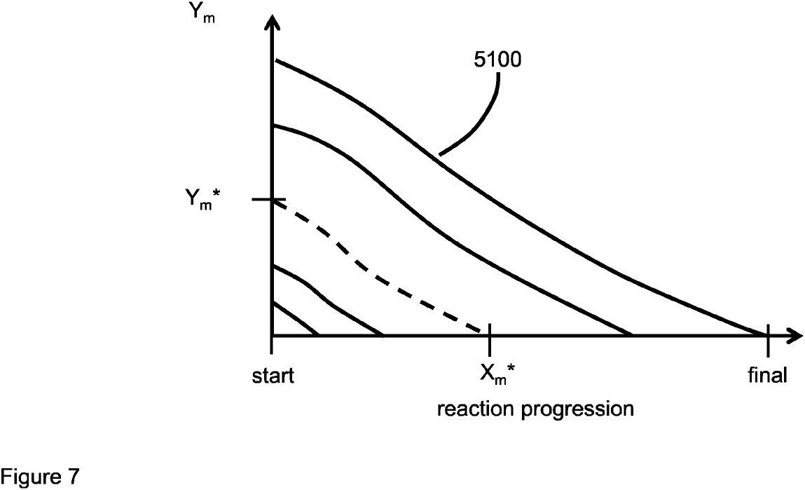

FIG. 7 illustrates trajectories along a reaction progression, the coordinate for reaction progression may be time or overall monomer conversion. In this example the reaction progression coordinate is overall monomer conversion. The advantage of this way of presenting the trajectories is that they can be used for processes where the polymerization rate is different from that of the reference polymerization process. Therefore, they represent an “universal” soft sensor which can be used as set points for any process. A selection of five trajectories is shown. Each trajectory crosses the reaction progression axis at a different point. Trajectory 5100 crosses the reaction progression axis at an end point of the reaction progression named final. For any instance along the reaction progression a respective trajectory may be calculated and provided to the output device. Each of trajectories containing the information of the movement of all polymer clusters in the multiphase latex polymer particles created up to an observation point on the reaction progression axis.

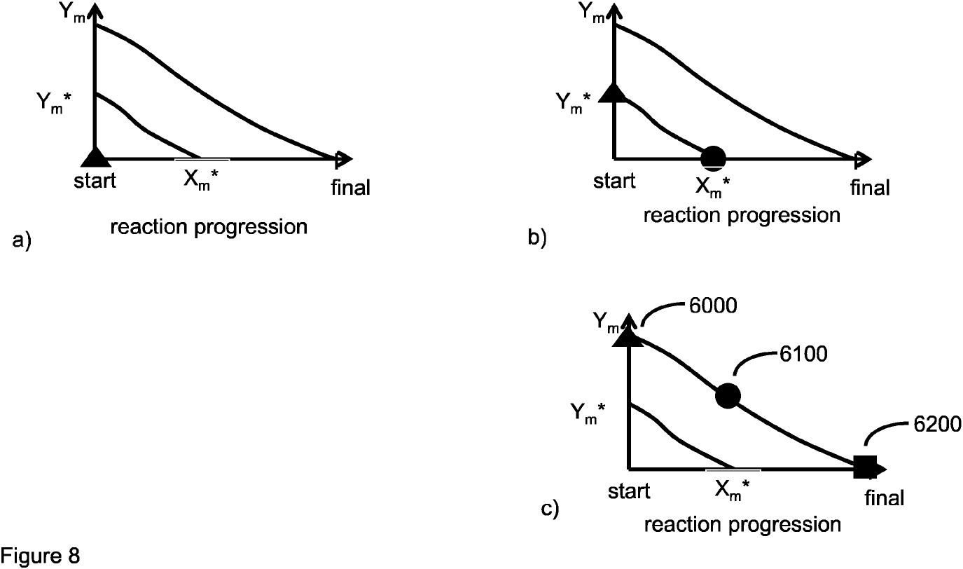

FIG. 8 illustrates the movement of polymer clusters created at different times during reaction progression. In FIG. 8a polymer clusters 6000 formed at the beginning of the reaction progression are represented as triangle. As these polymer clusters 6000 where just formed, no movement of the polymer clusters within the multiphase latex polymer particles occurred. At reaction progression of Xₘ new polymer clusters 6100 are formed, represented as a circle (FIG. 8b). One can see that polymer clusters 6000 moved to point Yₘ*, while the newly formed polymer clusters 6100 did not move. At the final point of the reaction polymer clusters 6200 are formed (FIG. 8c). All previously formed polymer clusters will have moved according to their respective instance of formation. Exemplarily depicted as polymer clusters 6000, polymer clusters 6100 and polymer clusters 6200. Polymer clusters are produced continuously throughout the emulsion polymerization process; therefore, it is clear that this illustration only arbitrarily describes the movement for selected polymer clusters 6000, 6100, 6200. A specific trajectory represents the movement of all polymer clusters in multiphase latex polymer particles since their instance of formation along a reaction progression, in the reaction process until the observation point, which would be the point where the specific crosses the reaction progression axis.

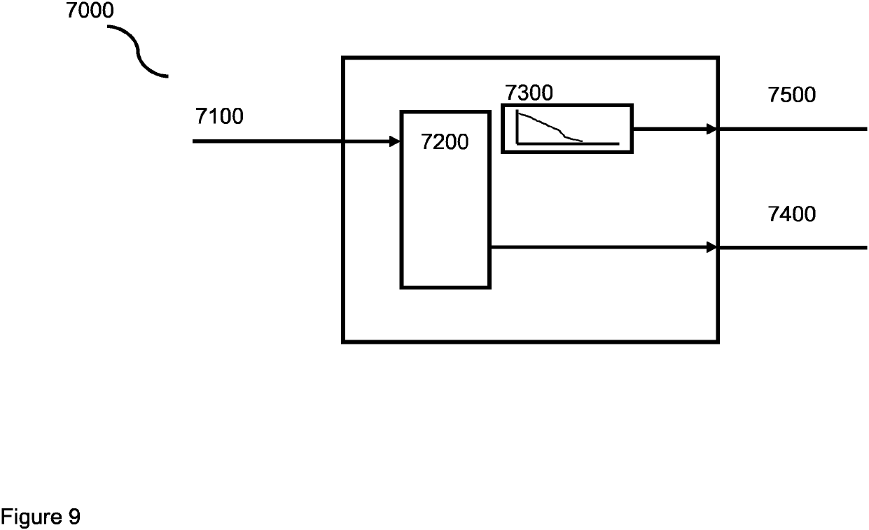

FIG. 9 illustrates a soft sensor 7000 according to the invention. The soft sensor comprises an input 7100, for receiving time series data from a production process, wherein in this example the time series data from the production process comprise a temperature of the production reactor, flowrates of each of two ingredients fed into the production reactor. Further in this example the time series data may comprise data suitable for determining the reaction progression variable of the production process, the quantity indicative of a polymer and/or monomer content inside the polymer matrix in the production process. In this example, the time series data comprises the temperatures at an inlet of a heating/cooling jacket and the outlet of a heating/cooling jacket. This allows to determine the heat transfer during the reaction. The soft sensor further comprises a morphology functional 7200, wherein the morphology functional describes the movement of polymer clusters in multiphase latex polymer particles since the instance of the polymer cluster formation along a reaction progression, in a reaction process, an output interface for 7400 for providing a reaction trajectory of the morphology functional. The soft sensor further comprises a reference trajectory of the morphology functional 7300. The reference trajectory of the morphology functional is derived from a historic polymerization process, leading to a desired morphology of the latex polymer particles. The soft sensor further comprises an output 7500 for providing the reference trajectory of the morphology functional. The soft sensor may be coupled to a processing device that determines the production trajectory of the morphology functional based on the received measured time series data of the production process. The soft sensor may be used for monitoring/ and or controlling the reaction process. The soft sensor may further be used for optimizing capacity of a reaction process. In an alternative, the soft sensor may comprise a single output for a deviation between the reference trajectory and the production trajectory.

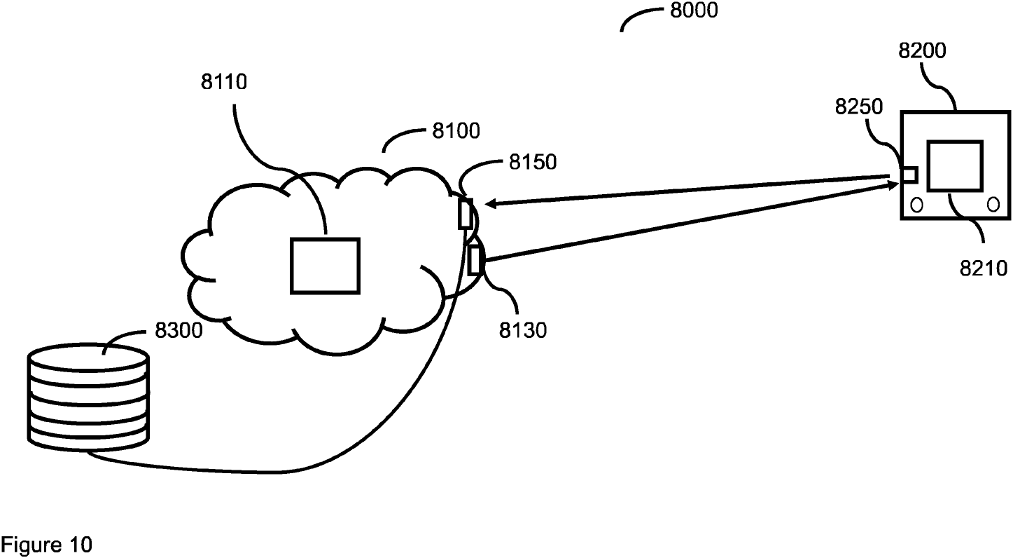

FIG. 10 shows a distributed system 8000 for distributing a soft sensor. A client device 8200 comprising a processing unit 8210 and a client device communication interface 8250.

The system further comprises a system 8100 for providing a soft sensor, the system 8100 comprises a database 8300 for providing a morphology functional, wherein the morphology functional describes the movement of polymer clusters in multiphase latex polymer particles since the instance of the polymer cluster-formation along a reaction progression, in a reaction process to a processor 8110. The database is in communication with an interface 8150 of the system 8300. Interface 8150 is further in communication with the client device communication interface. Time series data from a reference reaction process can be provided to the system 8300 via the communication interface 8250 to the interface 8150. The processor 8110 is configured for determining a reference trajectory of the morphology functional up to a reference observation point along the reference reaction progression based on time series data from the reference polymerization process, and the morphology functional. System 8100 further comprises an output interface 8130 for providing a soft sensor, the soft sensor comprising the reference trajectory of the morphology functional, the morphology functional and an input for receiving time series data from a production process. The soft sensor can then be received at the client device communication interface. In this example, system 8100 is a server, in particular a cloud server and the communication between the client device and the system is performed via a network. In other examples, the client device may be integrated into the system 8100. In one example, the soft sensor is part of a system comprising a monomer and the soft sensor. In that case tag associated with the monomer may be provided. The tag may be a computer readable tag, or an access code. The soft sensor may then be received by downloading the soft sensor to a client device. The download may be triggered using the tag. In such a case the tag is validated on system 8100, upon validation providing of the soft sensor is released. This may be by downloading the soft sensor to the client device.

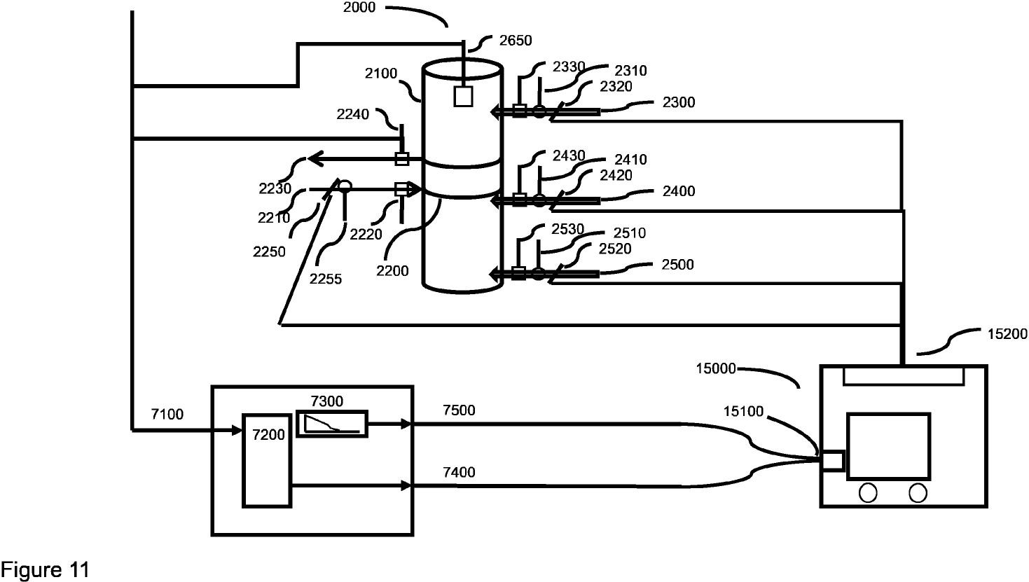

FIG. 11 shows an alternative system for controlling production of a polymer.

The system comprises a production reactor as disclosed in FIG. 1, providing measurement signals to the soft sensor 7000 described with reference to FIG. 9. Reference numbers corresponding to figures share the same reference numbers. The system further comprises a controller unit 15000. The controller unit comprises inputs 15100 for signals provided from the soft sensor, outputs 15200 for controlling the production reactor. The controller unit further comprises a processing device for determining control signals based on signals from the soft sensor.

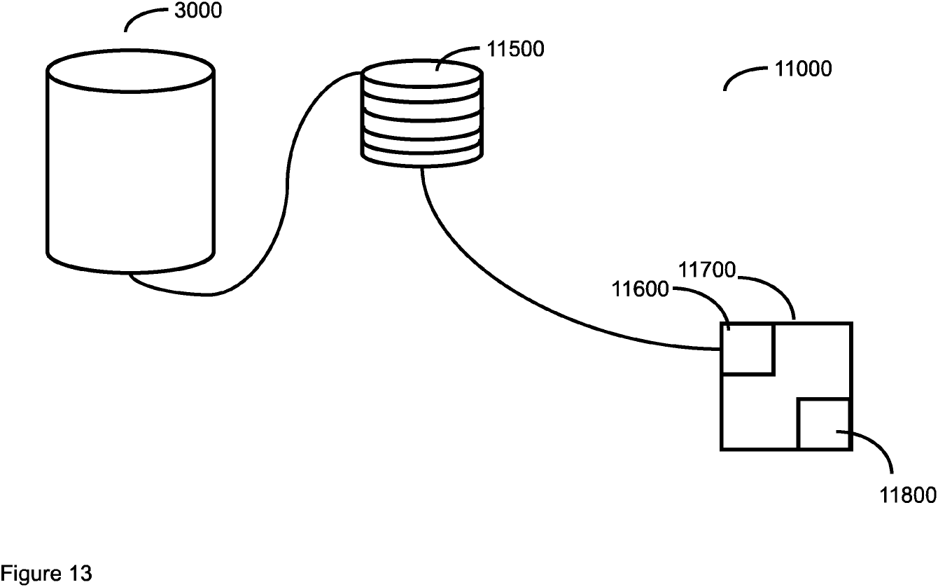

The input 15100 of the controller is connected to the output of the soft sensor 7500, 7400. The outputs of the controller are connected to actuators of the production reactor 2000, such that the production process may be controlled. The input of the soft sensor 7100 is connected to sensor signals of the production reactor. In FIG. 13 only some sensors from the production reactor are connected to the sensor inputs. This is for illustration purposes only, depicting all connections would render the figure unreadable.

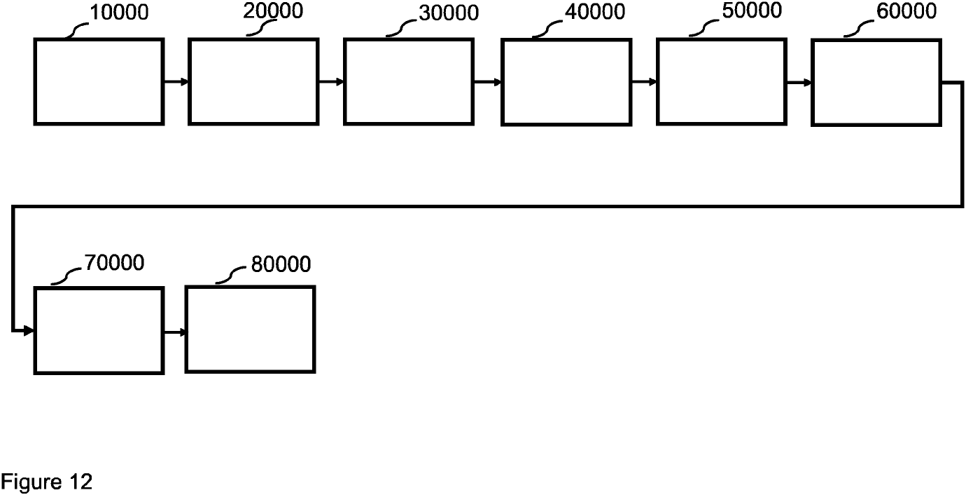

In a first step, time series data of the production process are provided from the sensors of the production reactor to the soft sensor, the time series data comprising at least a temperature of the production reactor, and flowrates of each ingredient fed into the production, at the soft sensor the production trajectory of the morphology functional is determined based on the time series data and morphology functional, wherein the morphology functional describes the movement of polymer clusters in multiphase latex polymer particles since the instance of the polymer cluster formation along a reaction progression, in the reaction process, The step of determining the production trajectory of the morphology functional works similar to the process described in FIG. 12 or with reference to FIG. 4 or the general description of determining a reaction trajectory of the morphology functional. The production trajectory of the morphology functional determined based on the morphology functional up to a recent observation point along the production reaction progression variable and on the time series data from a production process, may be understood as actual value input signal

The reference trajectory of the morphology functional and the production trajectory of the morphology functional are provided to the controller unit. In the controller unit, a setpoint value associated with the reference trajectory of the morphology functional up to a reference observation point along the production reaction progression based on the time series data from the reference polymerization process, and the reference reaction progression variable is determined. The reference observation point observation point along the production reaction progression based on the time series data from the reference polymerization process, and the reference reaction progression variable may be related to a production observation point along the production reaction progression based on the time series data from the production process. The setpoint value and actual value are provided to a control algorithm, wherein the control algorithm determines a value for manipulated variables based on the setpoint value and actual value.

In the controller unit a predicted production trajectory of the morphology functional is determined based on the production trajectory of the morphology functional at the recent observation point, the morphology functional and the reference observation point. The future reaction conditions expressed as the time evolutions of the amount of unreacted monomers (or equivalently the instantaneous monomer conversion Xᵢₙₛₜ), overall monomer conversion and temperature may be determined as

$$\begin{matrix}{\text{min}}_{{X}_{\text{inst}} \left(t\right) , X {}_{\text{overall}} \left(t\right) , T \left(t\right)} \left[{\int }_{0}^{X {}_{\text{overall,PH}}} W \left(X {}_{\text{overall,F}}\right)\right) \\ \left({\left({Y}_{\text{ψ,} X {}_{\text{overall,PH}}}^{\text{prod}} \left(X {}_{\text{overall,F}}\right) - {Y}_{\text{ψ,} X {}_{\text{overall,PH}}}^{\text{ref}} \left({X}_{\text{overall,F}}\right)\right)}^{2} d {X}_{\text{overall,F}}\right]\end{matrix}$$

wherein W is a weighing function that may be used to give more importance to certain parts of the trajectory. In an aspect, W(X_{overall,F}) = 1, for all X_{overall,F},

This determines the production trajectory of the morphology functional with the least deviation from the reference trajectory of the morphology functional. In terms of control, the difference between the recent observation point and the reference observation point is the prediction horizon.

Then, Xᵢₙₛₜ(t), Xₒᵥₑᵣₐₗₗ(t), T(t) are controlled by varying the feed rates of the ingredients of the formulation (monomers, initiators), and the inlet temperature and/or the flow rate of the cooling/heating fluid.

The control signals are provided via output 15200 to manipulate the flow rates and the temperatures accordingly.

FIG. 12 shows a method of producing a soft sensor according to the invention

In this example a first monomer and a second monomer are used. An initiator is also used. The selection of monomer and initiator is for illustration purposes only and should not be construed as limiting. The method is applicable for other combinations of monomers and initiators alike. Elements referring to the reference reactor are denominated according to FIG. 2.

At step 10000 time series data from a reference polymerization process, suitable for determining
the reaction progression variable
a quantity indicative of the polymer or monomer content inside the polymer particles in the reference polymerization process,
is provided to a processor.

The time series data from the reference polymerization process comprises the temperature of the reactor. In this example the reaction temperature (T) in the reactor is measured by the temperature sensor 3250 in the reactor 3100;
The cooling/heating jacket temperature; the inlet and outlet temperatures are measured in this example by the temperature sensors 3220 and 3240 respectively;
The flowrates of each ingredient fed into the reactor comprises measured data from the flow sensors for each of the type of monomers 3310, 3410 and the initiator 3510 throughout the entire course of the polymerization: ∀ᵢ, Fᵢ(t), 0 ≤ t ≤ t_{end}. Wherein Fi is the flow rate for each ingredient and i is an index for each type of ingredient; un this example the indices are assigned as follows (i= 1: first monomer; i=2: second monomer; i=3: initiator);
The temperature of the feeds measured by temperature sensors 3330, 3430 and 3530; the initial amount of each ingredient in the reactor comprises the initial amount of a first monomer, an initial amount a second monomer and an initial amount of the initiator;
the flowrate of the cooling medium in the cooling/heating jacket measured by flow sensor 3255.

From these time series data the quantity indicative of the amount of polymerized monomers in the reaction process is determined at optional step 20000.

The amount of heat produced by polymerization (Q) is then determined by means of the heat balance in the reactor using equation (2)

The mass flow of each ingredient is provided to the processor from the flow sensors 3310, 3410, 3510.

The amount of monomer converted to polymer may then be determined as

$$\left\{M o l e s o f m o n o m e r p o l y m e r i z e d\right\} = \frac{1}{\text{Δ} {Η}_{p o l}} {\int }_{0}^{t} \dot{Q} d {t}^{′}$$

Wherein ΔHₚₒₗ is the enthalpy of the polymerization reaction. If more than one type of monomers is used, data indicative of the amount of each monomer i polymerized (Mᵢ,_{polymerized}) and the amount of polymer can be obtained by using a modification of equation (3) as described in literature[Gugliotta, L.M., Arotçarena M., Leiza, J.R., Asua, J.M. Estimation of conversion and copolymer composition in semi-continuous emulsion polymerization using calorimetric data, Polymer, 1995, 36, 2019-2013].

At optional step 30000 the quantity indicative of the polymer or monomer content inside the polymer matrix in the reference reaction process is determined. In this example it is derived from the quantity indicative of the amount of monomer converted to polymer. In this example the assumption that the whole amount of monomer converted to polymer is inside the particles and that the remaining amount of unreacted monomer is also inside the particles is used. In this example, the volume fraction of polymer in the polymer particles can then be determined using the relation

$$\begin{matrix}{\text{ϕ}}_{p o l} \left(t\right) \approx \\ \frac{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}}}{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}} + \frac{\left\{M a s s o f u n r e a c t e d m o n o m e r s\right\}}{\left\{D e n s i t y o f m o n o m e r s\right\}}}\end{matrix}$$

In this example it is assumed to be the quantity indicative of the polymer content inside the polymer matrix.

The time series data from the reference polymerization process comprises:
the temperature of the reactor 3100. In this example the reaction temperature (T) in the reactor is measured by the temperature sensor 3250 in the reactor 3100;
The cooling/heating jacket temperature; the inlet and outlet temperatures are measured in this example by the temperature sensors 3220 and 3240 respectively;
The flowrates of each ingredient fed into the reactor comprises measured data from the flow sensors for each of the type of monomers 3310, 3410 and the initiator 3510 throughout the entire course of the polymerization: ∀ᵢ,Fᵢ(t), 0 ≤ t ≤ t_{end}. Wherein Fi is the flow rate for each ingredient and i is an index for each type of ingredient; un this example the indices are assigned as follows (i= 1: first monomer; i=2: second monomer; i=3: initiator);
The temperature of the feeds measured by temperature sensors 3330, 3430 and 3530;
the initial amount of each ingredient in the reactor comprises the initial amount of a first monomer, an initial amount a second monomer and an initial amount of the initiator;
the flowrate of the cooling medium in the cooling/heating jacket measured by flow sensor 3255.

From these time series data the quantity indicative of the amount of polymerized monomers in the reaction process is determined at optional step 40000.

The amount of heat produced by polymerization (Q) is then determined by means of the heat balance in the reactor using equation (2)

$${m}_{R} {c}_{p R} \frac{d T}{d t} = \dot{Q} + {\sum }_{i} {F}_{i} {c}_{p i} \left({T}_{i} - T\right) - {\dot{Q}}_{j}$$

Wherein m_{R} and c_{pR} are the mass and the specific heat capacity of the reactor and Fᵢ and cₚᵢ the mass flow and specific heat capacity of ingredient i that is fed to the reactor at a temperature Tᵢ. The mass flow of each ingredient is provided to the processor from the flow sensors 3310, 3410, 3510.

The amount of monomer converted to polymer may then be determined as

$$\left\{M o l e s o f m o n o m e r p o l y m e r i z e d\right\} = \frac{1}{Δ {H}_{p o l}} {\int }_{0}^{t} \dot{Q} d {t}^{′}$$

Wherein ΔHₚₒₗ is the enthalpy of the polymerization reaction. If more than one type of monomers is used, data indicative of the amount of each monomer i polymerized (Mᵢ,_{polymerized}) and the amount of polymer can be obtained.

At optional step 50000 the quantity indicative of the polymer or monomer content inside the polymer particles in the production reaction process is determined. In this example it is derived from the quantity indicative of the amount of monomer converted to polymer. In this example the assumption that the whole amount of monomer converted to polymer is inside the particles and that the remaining amount of unreacted monomer is also inside the particles is used. In this example, the volume fraction of polymer in the polymer particles can then be determined using the relation

$$\begin{matrix}{\text{ϕ}}_{p o l} \left(t\right) \approx \\ \frac{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}}}{\frac{\left\{M a s s o f m o n o m e r s p o l y m e r i z e d\right\}}{\left\{D e n s i t y o f p o l y m e r\right\}} + \frac{\left\{M a s s o f s e e d\right\}}{\left\{D e n s i t y o f s e e d\right\}} + \frac{\left\{M a s s o f u n r e a c t e d m o n o m e r s\right\}}{\left\{D e n s i t y o f m o n o m e r s\right\}}}\end{matrix}$$

In this example it is assumed to be the quantity indicative of the polymer content inside the polymer matrix.

At step 60000 the morphology functional is provided to the processor, wherein the morphology functional maps the morphology functional describes the movement of polymer clusters in multiphase latex polymer particles since the instance of the polymer cluster formation along a reaction progression in a reaction process, the morphology functional depending on the time series data, suitable for determining a quantity indicative of the amount of polymerized monomers in the reaction process, a quantity indicative of the polymer or monomer content inside the polymer particles in the reaction process and a reaction progression variable in this example the reaction progression variable is the overall monomer Xₒᵥₑᵣₐₗₗ.

In this example the morphology functional depends on the conditions inside the polymer particle. This dependency is in this example described by the cluster mobility function (ψ). Therefore, in this example the morphology functional relationship comprises the cluster mobility function. The cluster mobility function describes the mobility in the polymer particles during progression of the reaction process.

In an example the cluster mobility function depends on the data indicative of the reaction temperature, the quantity indicative of monomer or polymer content inside the polymer matrix and the glass transition temperature (T_{g}) and the molar mass (M_{w}) of the seed polymer. In this example the cluster mobility function is considered to be the matrix viscosity (η).

$$\text{Ψ=} \frac{1}{η \left({ϕ}_{p o l} , T , {T}_{g} , {M}_{w}\right)}$$

The morphology functional (Y) in this example is a functional that depends on the cluster mobility function (ψ) and Xₒᵥₑᵣₐₗₗ; Y=f(ψ,Xₒᵥₑᵣₐₗₗ).

In this example the, the particle matrix viscosity is described as:

$$η \left({ϕ}_{p o l} , T , {T}_{g} , {M}_{w}\right) = A {ϕ}_{p o l}^{n} \text{exp} \left(\left(B \frac{{T}_{g , e f f}}{T} - C\right)\right) \qquad \text{(9)}$$

Wherein T_{g,eff} is the effective glass transition temperature, and A, n, B and C are parameters that can be experimentally determined. Ways of determining T_{g,eff} and the parameters are disclosed in Properties of Polymers by D. W. van Krevelen, Klaas te Nijenhuis, 4^{th} edition, Elsevier Science, 2009. In this example, the effect of the molar mass of the seed is included in the parameters. In an alternative example a relative matrix viscosity is used (ηᵣₑₗ).

At step 70000, at the processing device a reference trajectory of the morphology functional up to a reference observation point along the reference reaction progression based on the time series data from the reference polymerization process and the morphology functional is determined. In this example, the reference trajectory of the morphology functional is determined, by

$$\left(\text{Ψ,} {X}_{\text{overall,F}} , {X}_{\text{overall,O}}\right) = {\int }_{{X}_{\text{overall,F}}}^{{X}_{\text{overall,O}}} \text{Ψ} \left(\frac{{M}_{M}}{{r}_{p} V}\right) d {X}_{\text{overall}} ,$$

based on the time series data from the reference polymerization process.

At step 80000 the produced soft sensor is provided, the soft sensor comprising the reference trajectory of the morphology functional, the morphology functional and an input for receiving time series data from a production process of the multiphase latex polymer particles, the time series comprising temperature of a production reactor and flowrates of each ingredient fed into the production reactor and an initial amount of the monomers of the production process an output for the reference trajectory of the morphology functional and a production trajectory of the morphology functional for the production process.

In FIG. 13 a system 11000 for producing a soft sensor is shown. In this example a database 11500 stores the time series data from a reference polymerization process. The time series data from the reference process may have been collected from a reference polymerization process in a reference reactor 3000. The data are then provided via interface to the processing device 11700. In this example the morphology functional is provided from the same database. In other examples the data bases may be different from each other. The soft sensor is then provided via output 11800.

1. A method of producing a soft sensor for a reference morphology of multiphase latex polymer particles synthesized in a production process, for use in monitoring and/or controlling the production process and/or optimizing production capacities of the production process, comprising:
providing via an interface to a processing device;
time series data from a reference polymerization process,
a morphology functional, wherein the morphology functional describes the movement of polymer clusters in multiphase latex polymer particles since the instance of the polymer cluster-formation along a reaction progression, in a reaction process, by relating a movement of polymer clusters in multiphase latex polymer particles since the instance of polymer cluster formation to time series data of a reaction process
determining at the processing device
a reference trajectory of the morphology functional up to a reference observation point along the reference reaction progression based on
the time series data from the reference polymerization process, and
the morphology functional,
providing the soft sensor, the soft sensor comprising
a reference trajectory of the morphology functional determined from the reference polymerization process,
the morphology functional and
a sensor input for receiving time series data from a production process of the multiphase latex polymer particles
an output for
a) the reference trajectory of the morphology functional and a production trajectory of the morphology functional for the production process or
b) a deviation between the reference trajectory and the production trajectory.

2. The method of claim 1, wherein the time series data from the reference polymerization process comprises a temperature of a reference reactor, flowrates of each ingredient fed into the reference reactor, and wherein the time series data for the production process comprises a temperature of a production reactor and flowrates of each ingredient fed into the production reactor.

3. The method of claim 1, wherein the respective time series data comprises the respective initial amount of each ingredient fed into the reactor.

4. The method of claim 1, wherein the morphology functional depends on a quantity indicative of a polymer and/or monomer content inside the polymer matrix in the reaction process and a reaction progression variable and wherein the time series data comprises data suitable for determining
the reaction progression variable of the production process,
the quantity indicative of a polymer and/or monomer content inside the polymer matrix in the production process.

5. The method of claim 1, wherein the morphology functional comprises a cluster mobility function, describing a mobility of the polymer clusters in the latex polymer particles during progression of the reaction process.

6. The method of claim 5, wherein the cluster mobility function depends on
the quantity indicative of a polymer and/or monomer content inside the polymer matrix in the reaction process.

7. The method of claim 1, wherein the cluster mobility function comprises a function of a particle matrix viscosity.

8. The method of claim 1, wherein the providing via an interface to a processing device the time series data of the reference polymerization process comprises providing the time series data via a client device, wherein the client device comprises a processing unit, and a client device communication interface for communication with the interface and/or receiving the soft sensor at a client device via an interface to a processing device time series data of the reference polymerization process comprises providing via a client device, wherein the client device comprises a processing unit, and a client device communication interface for receiving the soft sensor from the output interface.

9. A soft sensor produced according to claim 1 comprising:
a reference trajectory of the morphology functional derived from a reference polymerization process,
a morphology functional describing the movement of polymer clusters in multiphase latex polymer particles since the instance of the polymer cluster formation along a reaction progression, in a reaction process, by relating a movement of polymer clusters in multiphase latex polymer particles since the instance of polymer cluster formation to time series data of a reaction process and
a sensor input for receiving time series data from a production process of the multiphase latex polymer particles
an output for the reference trajectory of the morphology functional and a production trajectory of the morphology functional for the production process.

10. A method for monitoring and/or controlling the morphology of multiphase latex polymer particles synthesized in a production process, comprising:
providing to a soft sensor according to claim 9, time series data of the production process and
determining the production trajectory of the morphology functional based on
the morphology functional, wherein the morphology functional describes the movement of polymer clusters in multiphase latex polymer particles since the instance of the polymer cluster formation along a reaction progression, in the reaction process, and
the time series data from the production process,
determining a monitoring and/or control signal associated with the determined production trajectory of the morphology functional, and the reference trajectory of the morphology functional.
providing a monitoring and/or control signal via an output interface.

11. A method of optimizing capacity of a production process while maintaining a reference morphology, comprising:
providing constraints to a processing device,
providing the soft sensor according to claim 9,
determining with the processing device an optimal production capacity, based on the constraints and the reference trajectory of the morphology functional, such that the reaction trajectory of the morphology functional matches the reference trajectory of the morphology functional.

12. A System for executing the method according to claim 1, comprising a processor an input interface and an output interface, wherein the processor is configured for performing the method.

13. A computer program product, that when run on a processing device performs the method according to claim 1.

14. A system comprising
a monomer suitable for emulsion polymerization and
the soft sensor according to claim 9.
